SEQUENCE LISTING (16 sequences)

SEQ ID NO: 1 - DNA, 5217 bp, Homo sapiens
gaaactttaaaatatccctcagtgctcctgttaattcatggtagtgccccaaggcactctggcacccagttttggaactgcagttttaaaagtcataaattgaatgaaaatgatagcaaaggtggaggtttttaaagagctatttataggtccctggacagcatcttttttcaattaggcagcaacctttttgccctatgccgtaacctgtgtctgcaacttcctctaattgggaaatagttaagcagattcatagagctgaatgataaaattgtactacgagatgcactgggactcaacgtgaccttatcaagtgagatggagtcttgccctgtctccaaggctggagcccaatggtgtgatcttggctcactgcaacctccacctcccaggttcaaacgtttctcctgcctcagcctcccaagtaactgggattacagcaggcttggtgcatttgacacttcatgatatcagccaaagtggaactaaaaacagctcctggaagaggactatgacatcatcaggttgggagtctccagggacagcggaccctttggaaaaggactagaaagtgtgaaatctattagtcttcgatatgaaattctctgtctctgtaaaagcatttcatatttacaagacacaggcctactcctagggcagcaaaaagtggcaacaggcaagcagagggaaaagagatcatgaggcatttcagagtgcactgtcttttcatatatttctcaatgccgtatgtttggttttattttggccaagcataacaatctgctcaagaaaaaaaaatctggagaaaacaaaggtgcctttgccaatgttatgtttctttttgacaagccctgagatttctgaggggaattcacataaatgggatcaggtcattcatttacgttgtgtgcaaatatgatttaaagatacaacctttgcagagagcatgctttcctaagggtaggcacgtggaggactaagggtaaagcattcttcaagatcagttaatcaagaaaggtgctctttgcattctgaaatgcccttgttgcaaatattggttatattgattaaatttacacttaatggaaacaacctttaacttacagatgaacaaacccacaaaagcaaaaaatcaaaagccctacctatgatttcatattttctgtgtaactggattaaaggattcctgcttgcttttgggcataaatgataatggaatatttccaggtattgtttaaaatgagggcccatctacaaattcttagcaatactttggataattctaaaattcagctggacattgtctaattgttttttatatacatctttgctagaatttcaaattttaagtatgtgaatttagttaattagctgtgctgatcaattcaaaaacattactttcctaaattttagactatgaaggtcataaattcaacaaatatatctacacatacaattatagattgtttttcattataatgtcttcatcttaacagaattgtctttgtgattgtttttagaaaactgagagttttaattcataattacttgatcaaaaaattgtgggaacaatccagcattaattgtatgtgattgtttttatgtacataaggagtcttaagcttggtgccttgaagtcttttgtacttagtcccatgtttaaaattactactttatatctaaagcatttatgtttttcaattcaatttacatgatgctaattatggcaattataacaaatattaaagatttcgaaatagaatatgtgaattgttcacatacatagaaatgaaaagttcatttcgtaaagcaagatgctgggtgaaagagtgcttttgattgaaagatcactagattagtagagggcaagacttctagtccctaatctacccttaatagccatgtggtcacgtgtaagtcagtgaacccatctcattctcctcatacttttttcatctctaaaatgagggtataatttaagctcttcatttttttttttttttgagatagagttttgctcttgtcacccaggttggagtgcaatggcacgatctcagctcactgcaaccctctgcttcctcggttcaagtgattctcctgcttcagcctcccaagtagccgggattacaggtgcccgccaccacatctggctaattttttgtattttcaccatgttggccaggctggtctcgaacccctacctcaggtgatccctcgcctcggcctctcaaagtgctgggattacaggtgtgagccaccacgcccagcccaatatcagtttttcttttttaacacaaggctaacacaatcaaaatactagctaggggagaaaaaaaaaataaggcactgtttatgtgtaacaggctcttgttgcaatcactgggcagacaataaacagtaagaatcaatccttttcatatatccttcttgcagaatacataaaatcccacaaatggctatcttcctttttatgatatttggagaattgtagctaagtgacagatattttgcttgggtgtatagaccacaaaggactgtgtttgatgatggtttgcataaaattataccttagtttttactttgtatgttacatgttagatttagagtatgaaaattagtagggaggattattaacaaagaacagggcaagaggagtagaattaaacctcttctaatacctgtgcacaagtaggcttttcagaaactctacaaccctacataaactggatagttagaaaagcacactcccaaggaaggcggttatgttttgcagtttgaatcagaagaatagagctatagcaatcttcattctatagtaacattaaagagcctggtttatattatagcagtcattaagatttaaaaatttacatcttgccgttcttcttactcacagattttcgagaggtaatgtaatgatccacgaggtgagaatcactgccttttataatgcgattaaattgcatgaacaaagtttccaacaaataacagtaataaaaagaaacatgtattagcacttaataagccaggggctggacgacgtgtgttacatgctttcaatccatgaactggtaaactggtactagtatctctattggacatgtgaggaaaccaaatggagttgataaacagtagagttaaaaattactcttcatatattatattgcctcaatctcacagacatctctgctaccaaaagctatcatatctagatatgcggcataaggatgaccttggggcacactagaattctttgagagaattctggcagagaaaacaaatatttattcctacaataaaacccagcattttacaggttttatttttaactatgaagtattgttatctgtatctttcatataagtgtgcccggaatttatttcttctggtgggttcttggtctcgctgactccaagaatgaaaccgcagacccttgaggtgagtgtcacagttcttaaagatggtgtgttcagagtttgttccttcagatgttcagatgtgtccggagtttctcccttatggtgagttcgtggtctcgctgacttcaacaatgaagccgcagacctttgcagtgagtgtgtgacagttcttaaaggcagtgcgtccagagttgtttgttcctcccggtaggttcgtggtctcgctgatgtcaggaatgaagctgcagaccctcgcggtaagtgttacagctcataaaggtagtgcaaacccaaacagtgagcagtagcaagatttattatgaagagcaaaagaacaaagcttccccaccatagaaacggaccagaattggttgctgctgctgtggtagccagcttttattcccttatttggccacacccacatcctgctgattggcccattttacagaatgctgattggtccattttatagcgtgctgattggtgcgtttttacagagtgctgattggtgcatttacaatcctttagctagacacagagtgctgattggtgcctttataatcctttagctagacacaaaagttctacaagtccccacccaacccagaagctccgctggcttcacctctcgtaaggaaattgaggttcaaacaagtttcaaagtgctaaaactacagtttctcattctctgcaactggatttccactcatgtgtttgaatcccaggctctaagacttaacttgccattctgtgactttatgttcctgcaatttacacaaagctactatctgtcacatctctggtgttaacttcagactaaacttctttttgattcacaatgaccacacactttttggttgaggttttgctatcggtttattgtactggttaatagagagcttcttccagaaatttgagtagatggaagaggaagtagcacattcctaaaaatgtaccatgcctttcaagtcacaagcatccctatcacatggctgtcaagggtggctcagaataggtagagttaagaatttaaagtaaattggtgtaagcgatgaaagcttcatctaaaagcttatattacatcaactgaaatgtaaaataattggaacattttccaggcatccctgttatttatttgtctctctttccttgcttgcctacttcaaaagtcatatggcatggtgactagaactgtcctgccaaagagtttgtcaatataagattcctttctttgtaaacattctaccttggggcttcatttataatcaaaaggagtactgtaacctgtcaaaaaaaagctacctgtgacaatatattatgtgatggttacctgcagtaaggtggtggcaataaataaataaataatcacagaatgaaaccgagcagaactgtcagagaaatggtcagaattcacactctgaagaacacggctatacagtaataatcataataaatagccactcaatccaaaacatcactgggcgacttgtcacatatataatcagtggagatgtgattgaagcacaaggcttaagtgaatgtctagagagctaattgattcatttttatggaaattttacttattttaaatgtcatccctgaccatcttgaacttttacttgaagatttatttttttttttaaatcactgtttattagatttaggtattctggtctttgtttttcttttttatctatgtatgatttttatttttttatgcagtgtccttaagcttcatcaatgagaagaaatgtattaaaatccatttattcttaccctaaaaaaaaaaaaaaaaaaa

SEQ ID NO: 2 - DNA, 122 bp, Homo sapiens
atggagtcttgccctgtctccaaggctggagcccaatggtgtgatcttggctcactgcaacctccacctcccaggttcaaacgtttctcctgcctcagcctcccaagtaactgggattacag

SEQ ID NO: 3 - DNA, 5092 bp, Homo sapiens
gaaactttaaaatatccctcagtgctcctgttaattcatggtagtgccccaaggcactctggcacccagttttggaactgcagttttaaaagtcataaattgaatgaaaatgatagcaaaggtggaggtttttaaagagctatttataggtccctggacagcatcttttttcaattaggcagcaacctttttgccctatgccgtaacctgtgtctgcaacttcctctaattgggaaatagttaagcagattcatagagctgaatgataaaattgtactacgagatgcactgggactcaacgtgaccttatcaagtgaggcttggtgcatttgacacttcatgatatcagccaaagtggaactaaaaacagctcctggaagaggactatgacatcatcaggttgggagtctccagggacagcggaccctttggaaaaggactagaaagtgtgaaatctattagtcttcgatatgaaattctctgtctctgtaaaagcatttcatatttacaagacacaggcctactcctagggcagcaaaaagtggcaacaggcaagcagagggaaaagagatcatgaggcatttcagagtgcactgtcttttcatatatttctcaatgccgtatgtttggttttattttggccaagcataacaatctgctcaagaaaaaaaaatctggagaaaacaaaggtgcctttgccaatgttatgtttctttttgacaagccctgagatttctgaggggaattcacataaatgggatcaggtcattcatttacgttgtgtgcaaatatgatttaaagatacaacctttgcagagagcatgctttcctaagggtaggcacgtggaggactaagggtaaagcattcttcaagatcagttaatcaagaaaggtgctctttgcattctgaaatgcccttgttgcaaatattggttatattgattaaatttacacttaatggaaacaacctttaacttacagatgaacaaacccacaaaagcaaaaaatcaaaagccctacctatgatttcatattttctgtgtaactggattaaaggattcctgcttgcttttgggcataaatgataatggaatatttccaggtattgtttaaaatgagggcccatctacaaattcttagcaatactttggataattctaaaattcagctggacattgtctaattgttttttatatacatctttgctagaatttcaaattttaagtatgtgaatttagttaattagctgtgctgatcaattcaaaaacattactttcctaaattttagactatgaaggtcataaattcaacaaatatatctacacatacaattatagattgtttttcattataatgtcttcatcttaacagaattgtctttgtgattgtttttagaaaactgagagttttaattcataattacttgatcaaaaaattgtgggaacaatccagcattaattgtatgtgattgtttttatgtacataaggagtcttaagcttggtgccttgaagtcttttgtacttagtcccatgtttaaaattactactttatatctaaagcatttatgtttttcaattcaatttacatgatgctaattatggcaattataacaaatattaaagatttcgaaatagaatatgtgaattgttcacatacatagaaatgaaaagttcatttcgtaaagcaagatgctgggtgaaagagtgcttttgattgaaagatcactagattagtagagggcaagacttctagtccctaatctacccttaatagccatgtggtcacgtgtaagtcagtgaacccatctcattctcctcatacttttttcatctctaaaatgagggtataatttaagctcttcatttttttttttttttgagatagagttttgctcttgtcacccaggttggagtgcaatggcacgatctcagctcactgcaaccctctgcttcctcggttcaagtgattctcctgcttcagcctcccaagtagccgggattacaggtgcccgccaccacatctggctaattttttgtattttcaccatgttggccaggctggtctcgaacccctacctcaggtgatccctcgcctcggcctctcaaagtgctgggattacaggtgtgagccaccacgcccagcccaatatcagtttttcttttttaacacaaggctaacacaatcaaaatactagctaggggagaaaaaaaaaataaggcactgtttatgtgtaacaggctcttgttgcaatcactgggcagacaataaacagtaagaatcaatccttttcatatatccttcttgcagaatacataaaatcccacaaatggctatcttcctttttatgatatttggagaattgtagctaagtgacagatattttgcttgggtgtatagaccacaaaggactgtgtttgatgatggtttgcataaaattataccttagtttttactttgtatgttacatgttagatttagagtatgaaaattagtagggaggattattaacaaagaacagggcaagaggagtagaattaaacctcttctaatacctgtgcacaagtaggcttttcagaaactctacaaccctacataaactggatagttagaaaagcacactcccaaggaaggcggttatgttttgcagtttgaatcagaagaatagagctatagcaatcttcattctatagtaacattaaagagcctggtttatattatagcagtcattaagatttaaaaatttacatcttgccgttcttcttactcacagattttcgagaggtaatgtaatgatccacgaggtgagaatcactgccttttataatgcgattaaattgcatgaacaaagtttccaacaaataacagtaataaaaagaaacatgtattagcacttaataagccaggggctggacgacgtgtgttacatgctttcaatccatgaactggtaaactggtactagtatctctattggacatgtgaggaaaccaaatggagttgataaacagtagagttaaaaattactcttcatatattatattgcctcaatctcacagacatctctgctaccaaaagctatcatatctagatatgcggcataaggatgaccttggggcacactagaattctttgagagaattctggcagagaaaacaaatatttattcctacaataaaacccagcattttacaggttttatttttaactatgaagtattgttatctgtatctttcatataagtgtgcccggaatttatttcttctggtgggttcttggtctcgctgactccaagaatgaaaccgcagacccttgaggtgagtgtcacagttcttaaagatggtgtgttcagagtttgttccttcagatgttcagatgtgtccggagtttctcccttatggtgagttcgtggtctcgctgacttcaacaatgaagccgcagacctttgcagtgagtgtgtgacagttcttaaaggcagtgcgtccagagttgtttgttcctcccggtaggttcgtggtctcgctgatgtcaggaatgaagctgcagaccctcgcggtaagtgttacagctcataaaggtagtgcaaacccaaacagtgagcagtagcaagatttattatgaagagcaaaagaacaaagcttccccaccatagaaacggaccagaattggttgctgctgctgtggtagccagcttttattcccttatttggccacacccacatcctgctgattggcccattttacagaatgctgattggtccattttatagcgtgctgattggtgcgtttttacagagtgctgattggtgcatttacaatcctttagctagacacagagtgctgattggtgcctttataatcctttagctagacacaaaagttctacaagtccccacccaacccagaagctccgctggcttcacctctcgtaaggaaattgaggttcaaacaagtttcaaagtgctaaaactacagtttctcattctctgcaactggatttccactcatgtgtttgaatcccaggctctaagacttaacttgccattctgtgactttatgttcctgcaatttacacaaagctactatctgtcacatctctggtgttaacttcagactaaacttctttttgattcacaatgaccacacactttttggttgaggttttgctatcggtttattgtactggttaatagagagcttcttccagaaatttgagtagatggaagaggaagtagcacattcctaaaaatgtaccatgcctttcaagtcacaagcatccctatcacatggctgtcaagggtggctcagaataggtagagttaagaatttaaagtaaattggtgtaagcgatgaaagcttcatctaaaagcttatattacatcaactgaaatgtaaaataattggaacattttccaggcatccctgttatttatttgtctctctttccttgcttgcctacttcaaaagtcatatggcatggtgactagaactgtcctgccaaagagtttgtcaatataagattcctttctttgtaaacattctaccttggggcttcatttataatcaaaaggagtactgtaacctgtcaaaaaaaagctacctgtgacaatatattatgtgatggttacctgcagtaaggtggtggcaataaataaataaataatcacagaatgaaaccgagcagaactgtcagagaaatggtcagaattcacactctgaagaacacggctatacagtaataatcataataaatagccactcaatccaaaacatcactgggcgacttgtcacatatataatcagtggagatgtgattgaagcacaaggcttaagtgaatgtctagagagctaattgattcatttttatggaaattttacttattttaaatgtcatccctgaccatcttgaacttttacttgaagatttatttttttttttaaatcactgtttattagatttaggtattctggtctttgtttttcttttttatctatgtatgatttttatttttttatgcagtgtccttaagcttcatcaatgagaagaaatgtattaaaatccatttattcttaccctaaaaaaaaaaaaaaaaaaa

SEQ ID NO: 4 - DNA, 2085 bp, Homo sapiens
aggaagtcagagcgatgtgctgtgaaatctactaccgtttgctggttttgaaaatggagaaaaagagtgaggaactgagaaacatggatggccttgggaacgtggaaaagggtcactgaaatgggacgacatgaactcaaggagactatttatgaccatgtcatttgcaacatgaagaaagcttatctggagtgaaagtaaatgagaccaacagagatgagagacccggagaaatcctggttacactgcttgaatcctgtcagtcctatactggagtcctgttaatacaaaataatagtaataatccctctgtttcttatgtttatgccaacttcaacaaaaagaaacttgactaagagacaatataagaatttaatgtgtaattaagaaagaactctccaccacggggaatgtgaaaggtatatgagtcccttttcacgatgcgatgtcatgtcttttaaataagccatactttatgttcaataaaaagagaataagcaggattcgcaagagaacacaatccctttttaactgctgggaagatacttttagtcattaatgactggacgacaatttgggacacatatatggatattggccggtttgtgatgatgtgattgggcctctaagtgacaacattgttccctgtatagagtgagtggcaagtgcatttataaaattggccatcatggctgttaaatttatgagtctagaagtgtgcctctcaaacaagtatttgagaggttatcatgaagaaaaacaaaattaaaattattctgctgagctctcaaaggcagaactatcgaatcaaaattataggaaggaaggttcaaaccaacaataagaatgcctctcttgcattcctcatcactagaagtttctgataagatttttagaaaaatttagacagtattcttgaattgagcagggggataacacagagataattcaaatgtggtttgatgaacttctaaaatggttccaactataaacttcaattattctagcctccaagacctacacataactgtgcaatgtgcaaattattcaatagtaacatagaattctgaataattagcattttctcatggtttgatatttgtataaagcaatcttagcagtctgaataactagaatacttcaaagcaatatgttttattgctttatatttaaagaaaccatgtcagacatttaaaatgaatggctgctatcaggaaaatggatctcaagacacaagaaataaatcacaatgacttccttatttgtggaaattcttcttgtactagaaaggaaaagtagaagtatgggaaaatgtcttccagaaaattacattttcccaaagtttattttaaattttaataatttaatcaaccatctgattctgatttcaagtgtcacagaagttcattttattataaacaacaacaaaaaaactttgtagtcttgtaaacactggtgacagagaataacaccactaaccatgctgagcagaaaacaggagccaagtaagggaaaaagattctttatgtcactcatcaatttaaaagtgtttttcaccccaggaccatcctgaagccaaaactggggaaacatttactacttttacaaacaaaaatttatattccatcccttgcatgatatgtgtgtctccgacatttggtttctgtcttaagtgtctaaagaaaggccttatttagagcaaaatacatttttgtgggcattgatgtgaatttcaggtctgggttatcaatgacctaaattgagaaaatagaggggctgtgtggacaaggagagagaagagagcatacagctgccctatgccgttgctctgttcacagtgaggattgagccattctaatccccttggattttttatctaccaaaacagcttgggcccatcagcaaattgcagtggcatatggtccaggtttacgagtaatctggcacatgattaaggcaagggggatgtcatctttcagcaaatactgaaaatattgtagaagaatttggtagagtttttaaattcaaactgataaaattgtaactattaaatacttccccaaatcaaaaaaaaaaaaaaaaaaa

SEQ ID NO: 5 - DNA, 32463 bp, Homo sapiens
tctgagataaagagagagcctatgtgcttagtccaccagtgatggcagaaaggaatggctcatgaacctgtctacatgtggagctaaatggaaccaactcaacatcattccccctcccacttgccacactaaaaataaataaataaataaaagacttatatttctgagtcatgtttaaaattaattaaaaggatgtttgtatgagtccattatggcattaaaccaattccacaaagtagtctagggaaaacctcatgctatcagatccctcttgcgttctgcaatttctgaaaaaaagatgttcattgcaaagtgatatgagcactggaaaggtactaattccaatttgattctaattggatgagtgacatgggtaagcgattctaagcatttgtgttttttttagtagtatggaatttaattagttctcagtatgttagtgaagattgaatgaaaacatgcatatgtttccatgtattataaatattttaaaatgcaaaaaattattctaatgaatatataaatataaagcataacaataataatacaataccacccataaagtcatcatctaatttaaaaactaaaacattaacacttgaatctcccccattgcaacatctttcccgacttgtgtgtttttttcttttgcttttaaaatttttgttttatcatatgtctgcataagattatatagctttccttgttttaagctttttaaataatatattgtagttatattatttgtgctttgctttttttacttaacattatggttctaaaattcagtaatgtgttgggcatgtataatttgtttatttttaatctctttgacattcgactatataaatttcagtttgtttattgactcctttgtctatagatactctgctatttctgtttttgctgttacaaaaataatgctgttttaaatttcattttgtatacttttttgaggcatgtgtatgagttattctaaggtaaaaaaataagaaaaaattgctgggttataagattgtcacatgctcgaatttacaagataatgccaaatcatttttcaaagtaattatacctatttatactaccggtatgagtatattggtgcccacatagttgcttgttctgccaaagtttggtatgatcgaacaataatttttgcccatcaaatggcataaaataaaatctcagtgtgcttttaatttgcattttctatgtttaagaattgtttcttttttaaccatttataatttacttttgctgaaatgcttgcttattatttttgctccccattttttcctattggattgcttttctcattaatttataagaattttatatggtttagatactaattattatattactgaaaatacctttatcagtttgttgtgtactttctactttatgtcttgtgatggataaaagttttaaattgtattgtgttgaagttaacatttttaaattttataatcagcatctttaataatctctttataaaattttcctttacatagatgtcataaagatacatctctataatttcttatttttttggcatatgttcattaagtcattttatcattttttagtaataaattgcagttatttatgaaacaaataatttttaaaattatatatgctttctttaaaaattgatcttagcatgcttcactatgaagcttgaggcttcactgcacgttgtactgaaattatgtataaaacagtggttctgaaaatctctgagttcatgacacctttagtgtctcaggtttttttgcttttgttcttgttttttctcacaaagcacctaagttaaataaaaacaaagcacaaagctatcagcttcatgtattaagtagtaagctcccatgttaacagttgtaacttgcctggtgcccaatagatgtcactctgttttcctagaaactttaaaatatccctcagtgctcctgttaattcatggtagtgccccaaggcactctggcacccagttttggaactgcagttttaaaagtcataaattgaatgaaaatgatagcaaaggtggaggtttttaaagagctatttataggtccctggacagcatcttttttcaattaggcagcaacctttttgccctatgccgtaacctgtgtctgcaacttcctctaattggggtgagtaagagattttgttatgtatataatagctaagaatatagtaataatggcttaaatcatggttatttttaaactactaacatttagaagacaaaataaaaatgctttgaaaagtatagaggttttagtgtaattagcagggaataatgaaatgatttgatagggctactcagttttgtataactttggtgctttaagtctgaatgcagagcatggatgttgtgatccagcctttatatgttttccctgaagaagatttaatttatttggccttttgagaaacacatttggcattgtaatatgttttgcttccaggttctatctccaaggataatttgacaaaatcacacataaatttattttcagggcacacagtttcccttttagggaactcacagaggtagagagtaatacaataatcacatttgaatattcagtaagtgaggtcctcatagatcttatgtgtatgtcaccatgtatataattttgttaatcactagatgtatgagacaagaaatttgaggaaccttaactagagattaaaatagggatttaaatcaaagaaacatttaaatgcctcctttattatttaaatacctgcatggtgaatcattgaaaaaaaaataaaaagcatacaacttgggaatattctaaaccaagaagaatttgttattctggttgatttttttttttcaggctcccacaggcaacttacctttatctctttgtgatttttatttcttgttaaaatatacagaaatagttaagcagattcatagagctgaatgataaaattgtactacgagatgcactgggactcaacgtgaccttatcaagtgaggtgagccattcattaattcagataatggaacttattatcataatcttttgcttatgctattgttgagcttaactacttattcatatttgcatatgcatattgagataatatcatttcattaatttcagtactgaacactaatctcctaagagtaattgtgaaagtttcagattgcactatttttaactatatatctgtatgttatcttcatatatgcttgaataacttataagcaattgaaactttcaattacagtatactattgaagcaaatcaactaatatatacacatatccattagcaatagtagataatttttgtaaatgtccagcacagttcttcatatgtagaggatgttcaaattggctaagttccttttctctcttaattattagtatttttcctactgctctttgtataattattccttcctctttagctccaatccttacaatctattcttaacatagcaactggaagaaagtttttaaacataaaccagatgatgtcactccaccccacaaaacttccactattctctgtcacacatagaaagaaagaaaaaaaatattgaaaacctacaaagacttgctatgatctggtccaggctctccctaaaatttcatgtaatttccagccactaggcctttctggctctccttcaatctcattagccttttcactactacaagttagactgggttttggccgaggtatttctttttttcatattttgcctttgcctagattgctcttccaatagatattcacaattgcatcatcatttctatatacgtgctaaaaggtttccttgtccaaaatagcttcagtgaccacctgatctagaatagtctcgatcaaaagtttcttttccttttcctcaccacttgatatttatatcaaacatttatttgtgtaatttatgtgtttgtttgttttctgtactagcattatgatgaccatactatttgatgccccccaaaaaatactttcgagaatgacagggaaagctaaaataattaaattatataattttgacataggcactattgacaaaaagcaattgatgttatgatagtgttagatctatgaaatagtactatttaaaagtaattctctgaaatacaattttctaaaactaaaagcagcatatgtacatgaaacaccaaaaaacttccttatatttatcactggaagatttaaaatagtataagtagtaacttatttaatatatttttgattatttaattaattttatagtatccaactctaatataatgccagtggtatttgttcaaaatattttaatgttgtctatttatttttaatttgcctaaaaattatcttaaatgaaaatttttggttaataaatttgaaaatactgaaaccctcatctcagtctctgtggatcctaaagtttttagttgagaaaataatttttctctagagaatgaagtagcttgtaagcttggagaaatttctgctaaataaatgatattatcaactctattttcttcaatacgaaatatataaatatttcagctcatatatttttgcaggtgctatgcttttgcttccaatcataatttctgacaaatattttggaagtcaaaacttgtcttctattttgttatttaaaattatatagactacttttgtaaacctttatactatcaaatcataggcaatttcagtttgatttcattctggtgcagaatataagtttatccaagtaaaactggagtcacttcaaaagattcctcccactgactgagatattccaaagccaactttgcaaaatttcagaattaaatattatacttctttgtaccttcattttatttgttcaatttttctttgtgtttgtagaaaattttaatatttttctgttttcaagttttgattttaatttactactttataatttttaaaggtaagttttgtgaggctatattcattatgtgttttgaataaagacatacaattaattttgagaactgcaataaaaattataagactattaaaaatgcagtaagtgtactacacttaggctgctaaaaatgcagtatcagtagactacatttaggctgcttaaagttagttcttctaagtaccatatactttaaaattttagctaatgatggagaacaaagacagaaagactgtgttaccatattctagttggccattttgttttgttttgagagacgtcacatcagccttatcataaaaattatttggttttaccattttgactgtgagcaaaatatacagcataatatacaaaataaaatatatgtacatcttcacaacttcttgtttaggatgcaattatatatatatatatatatatatttattattatactttaagttctagggtacatgtgcaccacgtgcaggtttgttacatatgtatacatgtgccatgttggtgtgctgcacccattaactcgtcatttacattaggtgtatctcctaatgctatccctcccctctctccccaccccacaacaagccccggtgtgtgatgttccccttcctgtgtccatgtgttctcattgttcaattcccacctatgagtgagaacacgcagtgtttgcttttttgtccttgcaatagtttgctgagaatgatggtttccagcttcatccatgtccctacaaaggacatgaactcatcattttttatggctgcatagtattccatggtgtatatgtgccacattttcttaatccagtctgtcattgttgttggacatttgggttgcaattttgagtttcatgtgtagcatgtatagcacaaccaattaagatttctttctttctctctttttttttttttttttttgagatggagtcttgccctgtctccaaggctggagcccaatggtgtgatcttggctcactgcaacctccacctcccaggttcaaacgtttctcctgcctcagcctcccaagtaactgggattacaggtacccaccaccatgcccagctaattttttgtatttttagtagagatggggtttcaccatggtggccaggcttagtctcaaactcctggcctcatgtctgcctgccttggcctcccaaagtgctgaaattacaggtgtgagccactgtgcccggccaacatttcttaacctagtagtttagtttttatagttttattaccgcagtgaacgctacaccaaaaatttcatttgaattattaacataaaaagaaataattttgtattaaacttttaaactgaattttcaatagcattcacaatcatattaaatgtttcaccttttacagaataaacttccatgagctttatttgttaacatttattgatacacacaattaataaagcatgtaagtaataaactgaaattacttaactggttgagaaattttttttttgctaggagaaccaacacattaagagcagtatcttcaattttcatacatgaacaagaaaacttcgaagcaaaaatgaatatatttaatttagaacaatgatttttatttgaaaagtcacattgcacagagtgatatataaatgaattttctgaagatatatgtgttaaatcagggctttcaggcacagtctgcttaaaactttggaaagagatactattttttttcagtgcatttgtatcttctaattttctcatagtaatatcacagggtccccataggtgatgctgaatatgggcaactggttttttttgtttttttttttttacctgttgtcttagcattccctaaaacaggggtcaccaaatcccaggccacctagtggttctggtccatggcctgttaggaacgaggccacacagcaggaggtgaacggcaggtgagcaagaattaccacctgagctccacctcctgtcagatcagtggtgacattagattttcatgagtgtgaatcctactgtgaactgtgcatgtgagagatctaagttacacgttccttatgagaatctaatgcctgatgatctgaggtggaacagtttcatcctgaaaccatccgcctgccctgtccatggaaaaatcgttttccacaaaaccagttccttctgccaaaaaggttggggactgttgccctaaaacacaaaccctaggcaaaatttttacagtaactattttattgattgtgcaacccaagaaattcttgaggttgggaagaggatgagaagttagtaggggaaggagggacaacaaatataaggagtggcattgttagacaggtcagaactttgagacaattgctctgtctagtgagccatcttcaaaggggttgcttctctgactgtttaccttgggagaagcagggagaaaaataatatctaaataatatctaaataacactgaaggcttgtctcatgccttcagtgttcaccccagacagcattaattactgcagcatcttatacttcagcagtattcaggaacccagggaaagaaaaggaaggacttccaatgtaggttgaaagtgaggcacccatagggcatatccagcaaggttttcagtctgttgaaatgagagtgggcacagaatgggaatattaccagccactgagtgccacccaatttagaaagcaaagcaagtgtgcagagatgggtaatgcataatctgaatttggtacaatccattcatagtgcaggtcagatctgctcacactgtactacgatattcaattctcaccttcgattcttccctgagaagaagcataaaaatttaatctttaagtattattgttaactgctgcctccttaaacaaacagcacacagacaactttatattatgttatccaatgaagttacagctggccaccttcagctataattttgggtcttgctgaatggggtgatccagaccttggatttaggggttattattcctaagttactttgagcttctggacatgtgttctatgcggttaacatttttttattatcactgaatacaaaagcaccaagacatatcccagtgaatctactgggttccagacatatttctttttgccctcactatgtagtagcaactatagcttctcatgattatcaagatcgatgactcccactagtacattccatctgcaccaggattaggagctcaaggtagttcatacatttcttctccacttggaaaataacatctctaaactagcaaagtctaaggcagggtggaaagcactatattttgagcatgggaatatgttaaaaataactgggaataatggcaagcaggggatcccacttctactccttacctcccagacctatgcttcagtgcatatagtgtacactagaagacaacaccctgggattttttctctgagctggctccttaactgagtcttcattctatcagtgtagtagaatactagtagtaagccccacgaatcttgtgattacctatgcatcaacatacctcctttgtgataaagcaggctccttgttctaaagcaatcccacatgaaatgctctgttgagatatcaggcattttttaagcccttaaatgatggtgttgattgaaaaactgtgcaaaaaaaggcaaactcatatccagaatgtgcatttgttctgataaggacaatccttattccctttctggtggaaggggtcttatttcatagctctgtcaccaagaggctgcttaaactcccgaaaagtagtgtcacatggaagactctgcaatggatatgcactgctctcagactggacattcagcaatggtagcaatagatcagccttggtgaagacgaatacatgaatacatgtcattagcctcatgcatagccttaatccttctgctatgaccaccctggtcatgtgctcattgtgcaagcattgcaatagtcatcctgtctaccttgttgaagagatgctccctggagatgggttctctctggtagaaattaatactagttacacttatctgcatgtgttgtgcccactcctataggatcttccatatacttccttcccagagttctttagccccgacttattagtctttttcatttgaggactctgaccattgagtctggccactgccacaacccaggaatcaacatattatctgtaccttttgtcatctcccatttcatgcaaaatgaatgactgagttgactgcttaaaactctgctcaccaggagaagttccctttgttgctgtctctaaggccaaccctgagtggggctgaaatgtggtagtggtctacttcaagtttgcaccaacattttgggctgtctcttctgttaatcagatcagtttcttcccttctttgatgttgcacacgaggaacttcctgttgcacattttgccttctatgattgaatgtcatttactaatttataatatttattgcttttcccttctatgattgtatgtcatttgcttatttataatatttattgcttttccctttaaaacataatgtaagctctatgagggttactttttcaattttttattaactgataggtcttgcttgcctaaaaggacctggcatatagtgctgcatacattcaataaatgtttggtaaaagaaagaataaatgaactctctctatctcctttccctgcttcaattttgttcatagtatgattcactatgcaacattatacattataatatgtttttttttcagtctttccctcctccactaaaatataaaacacacaagaggttgtctgctttgtttaaagcattatccccaacacctatagaagagcctgctttgacaaatactttttgcattaattagcaaatagacaaggtttttctcattccctgtcaccaggtaacacctaatgccctgtaggttatataacaggcgctcagtgaaaaagttgtgatacaaaataaatgcaccaaaattttgcctctggcccagaggtggcctctctcaatactactctgatctttcagaagtagaattgacaagaactaacaagataactttgcctccttaaacttgttcctcaggaagtttcaaaggaaaattcaaataatttatcaaccaaaaaagactagaaattctcattttttttacacacatagcttttattttgaatcctgtcgatttatagtgtaaggcttccgtgattcctattaccttatttcattgggaaaaagaaacaaaacaaaacagaaaaaactggaagccaatatcttctgaagtgatgtgggaagctaggaggctcctttgttacctttacttcacctcacatgtataaagaattccattcttaaaggacatcacattttttaaaacactgttgtattttattatatgcatatctggaatagaaaatctgacaagagggcagccctgaacaattttccctatcattacgtagaagatgaatatggagttaattttcacttttgacacattatattgagattttaaaattcattctgtctaagttgggtttggaagaagtctaatctcacagtttcatttgaggacagctctatgaatatttgtgaaaaatgaatattttatttgccttgaaaactatgattcaatttgatgaaatgaaaaaaatttatggataatgtttaatcacgactgtcatataagagctaagctgtcagcaggatgctatgacacctagagttagttacttccagctgagagaggatccacacagcctctaatagcttaggaagctgtgacttgatattctggctcatttaatagattcattgtgcttcatgttctatatgaaattctgtttgaaaccagcctaaatatgttttaatgtttaatgtcattatattctttatacatctactttttaaaatgaatgcagaaatatttttccaaaatttaaaaaggcattaattgacatgaacatcatttttatcatgaagcttaaagagatggtttgttaagttaatcaatatgtgatcactatgataatgttatatactcagtcttttgctgggaactatagagttatcaacaatatatggttcatgtcctctactcctgaggaattcctaagtggagagagaaaaactatgtaaccctacagtgtaatgtatggaacatacttacttgtgaatgtatgtggcaaagactaggaacagtagcactgattttaagtacaataattaaataaaagtgattattttgcttttttttctccttaatgtctaggaacttttggtattcttcccttccatttattaagctacatgctcattatttccttgtttcttagcaatagccagactttaatccaattggtcatttactactttcaattcaatgtgattcttttactcttctcctactcaatatttttcaatattttgacaaaaatttagacaatatgcctattaactttgtagatgctacaaaacacagtaatttttacattttataacaaaaatcaaatttaaaaatgaacaagcagtttgcagagatttaaatagcaaatgcacatttaacaagtataagagtaagtttggtacttggttacaaaataccaatttcacatgtaaataaaggattttttaacaatagcattgtgaaaaagaattgaacctgagacctcatattcagtttaagactatgtgatattttgtccaagaaaaaaataaatatgttatgcaaatggaaactgagtgtttagaataccgaggaagataattctgttctactatagactgcatacgttatacctgaattcttatgtttggatatggactccctactttcaaaacacaggaatatagtattattcatttagaggctcgatattattattataaaatgcatggttgaagaaaatataagtatacaggttgaaaaagaaaattagaaaacactctaagcactgaactacaattttagacctgaataaggaaaacttttttcaagagccagaaatattcaaaaactgagttggctactggaagaaaatactaatgtagtggtcttggggttttcaaacatgcaccagaaaaccaaatactagatctgtattgtagaagagatttgggcaatgtttagttagttgaaataaatgtccttaagtatgaaattcagtgagtctatcatatcttttatacatatacatatgtatcttttaattgcccccaaccaacaataaatatgtcagttctcatatcaaaactgcaatcacttcttcatctatttgctttttattatttttgcataacatttttaaactgtggttttcaaaactatagttacagggtagaacactaatacacttctctcttccaaaaccccctttgtaatttattatgtgaagtttgttttccttataatacacagtctttgaaacttagtttccacacttatttatttatattaacacttgagtcatgctaactctctaatcactggtattcaaacccaaaatttaaattgttgcttttcttagcctatttcacctccccaagtcctctgtctctatattcaaccacccttcaagacctatcttcaattgtataatttccagaaagactttggggatactgacactaaatttttttctcatttttgaatctctaccatgcttaataaaacattattaccacgctatttactttcatgtgcagatccttgagtaaaaaattatccctgaacttcatgtccagagacctgggttctacttatgactgtacttccatttagctgtgtagcctcaataaagcattgaaactttctgtatgacaggaactttatccatacaatgcagatatatttcaattataataaatattgaatgaatctttttgcctcaggatcattcttatattgttgacttgtttaattattataatataaattcatcatgagaagagtggtcttccattgccttgtttccccataatacttatcatttaactattcacaaaatgtccacttgcatactttcaataagcacaaggtcaaataaatttaaaactaacaagtattttatagtttcattcactctttttatttgctgacactttgatatattttcattcctcaaaataatttcagtatcttgacttttaaatattttttcaggatattaatgagatgtctcaatcttaaatattgttttccttataaatgctgtgtaaagaatatattttatgcatcttaaaattatccttacagtataaacaaaggtttatttccctaaacttatgaccaaactgagcaaataagagaaatgcaatgcactattttgctcattgatcacatgaattgtctccagctttgaatctctgtcctcatttattttttatttttacttatttacttttgagacagggtctgtgggtcttgctgtcacccaggatggagtgcagtggcacgatcatagctcactgcagcctcaaattcctgggttcaagtgatccttctgccttggcctcagagtagctggcacatgggaccacagacacacaccaccactccctgctgattttttttttatttgtttgatttttagtaagagacaagttctcactatgttgatcaggctgatctcaaacccattagctgaagcaatcctcctgcctcagcctccaggtgctgggattacaggcatgagccactgcacccagcctggccttactttttaattcagaaattaaatacaccactttgaaacaagataatatatcaacctctagtaacagatttcttctgaaaaggtgttcctaacaaaacctggaaatgaccataccctggcacctgctgaggggctctgcctgtcaatggcatggcttcaactggaagtcaatgataacagatatgtcattatccctatgtgatatccaggatctaaccctgtccattctagttgcttctgatgttccaccttagaataattgaattctaatggataatgaccaattacttttcactcattagtttgagaagtggtgtcctcactcacatgaaacggagagcttgcatgcttatgttcaataaactaacaggcatggctaccagtcaccactactaaggaggagtctttgtcaaattagaagtactatttcagggatttgtgcatgtagattcactgttgaaagaaaatgatctattgtcattccagacatcccatcttgcttctgcataatattttcagactacagaaaagaataaagagaagatagatctctttagtgtgttgttcttttacagctgcccaatgctaaagcagatttcaaaagttgcaggctatccttttttttcccaaacataattagagcacttagccaaacatgtatgcatattgtagctgttgactattgccgaaaacacaaaatttcagaggcacaaaatttcattggcatacaaaataaacatttgctttttatccatccatggtcaactgggacagctcattcaaatcccactcttggaatggtgccacaattccactggatggctctactttcaggtctgagtccacctgagcttggttagggaggcttggcttcacgtaagttcattattcctgaagggatagcagctaccccagggtatcttttctcacaacgataacagagcaagagggaagaatgagatgtctttgaaggcttagactaaaaactggtgcactttcacttccagcaatattttttgtttaaaaagaaatcaaatcaaacagcagtgaagtaaagggacaggaacatgtactccaactttagtgggaggaagttaaagtgtgtgtatatggtgtttcttaaagtttaaagatccttttgataaaaacaacaaatcataaaaacactctagtagatgaaagacagaattcttttactgacaactccaaatttttcaggcagggccacctagggagttgcatccagagaaagggtaacagcaagctgaagctatggtggggcaatttatataacaagtagagtggattcaactagcttcccaggatccttgtgcattggctaacttgaacaatttctcaggctccagggcttagggagtgtgtctagttgcctgatacctggccctggggcaactgaggagcctacatagtggccaagagtttgaaagctcaataaggggagtgattggcttggggagtggacttaatcagctgccaagaagggaaactgactaccctctagcctgggcctcaaaactgggtccagacagtgattaaacaacagcagaaactgttacaatctaccgcttaaagtcttgacatcagttttgagcttatttggcatatttcaatggccaaatagtagcaaagactttcacagagtagagtataaattgtccaaagagcatagaagacattttattatgggagtaaataggagaaaaacatatagaagaataaagagtccttgtaggccagtccatcaccaagatctccattttatggcattactaagaaaacagagctggaatttctggggtccctaacaagatcttggtggagttgtaaaatgttttaaaagtaccaattatgtggttttatgataataatatcttgatgtatccttgggtcaaggtggtagttccagtagttaccttgtagagggtatacagttagtaaagcatatgtaaaggttagtgatatttgtttgaaaccaagtgaagaggtggttgtggtcatgttcagaaatgcagagtgggaggtcgtggtggtgagttgaagtgggaagagtagggagaaggcactgggaaatgggaagttcactccccaagggagtcaatgggtggactgatatgaggtagtttcctttctggggaaagtagtaatcttctttaatgtttagaaaggcacagatgctagtaactgctacaagttcttccttaaggcataggagtttaggagtgtatgtttttacagagagccatgagtacacacagaggtcagatatatgtaactttcaaaatagccaagggaattgggaacaatatttaaaattagtgttacatatgttgggccttgaattaaaaaattaaattatggggcaagatacagatgcatttaggaattcatttgtcattttgtaacttatataggagttctttgataggcccatttaggtctcccaattaaattagctgtctgggacctatcccagagagaaaagttccattgaatgccttttgtaagagcccactattgtgtattctgaaatgtgaaatgtgtggcttagtcactgtcagtaatgtctgacacgtcaaagggaataagaagtatcccaggaagactctgtattgtggccttagtataaatagcaacatgtgtaaccttgatatatattttggatacttgtgaagcaagagattctcaaagttctttactctgaaggggacaactctcaggcaatgtttcaaaagtttgcaaaaatgtggagatggagccatttgctgactatgatatctagtgttaagatgactacctagtttgggcaagcgacatgacccttgggtttcattctttatctggaacattctgggtaaaaaatggaaagcagcaatatttccctgtgctccatcatgtatgacagtgacaatgctattgtacactctacaaactcccactgctgttcactcagttaatcataagtggctctcagatatctaaagggcttggaagaggggtaaacttctccaacaccatggcatctggcaagcgactgaggacattggaggccatcccctcttgcaagtggaatatgccagagggctgagatttactccattctacatcgagaagtcctttgtagctgtgcccaacttgtctggttctgcttctgtgactcaaaacataattggcagcttgatatgaagagtcacagacttaaggtctgtaaaagcctctatttccaggagataccagtatgtggccagcatgttcctctctaatggagactagcacaagaatgagtaggattggccaatttgcataatttcattggctcagggacataaaggctgtcactaattgtctgatagctggccctagagtgatttaggcaggtgcttactggtctcaagtgtgaaaatacagtaaggcaaatacttagctgttcgaaagggggaactgaccagcctctagtcaaaaccacaaaactgggtcaagatagcgtttcaaataaactacataggggtatgagtgaacatttggagccaaaaaatcaatctgttatacataggcactactacaaagattcttcagtatccaaattattataatttaagctgaagagtgtgttggggatggtggtagaataaactcaccaaataactaataactaatggctactaggtaacagaaactatgtgaaacactgagctgaaaatgactgctgcttaacaatgagatctttcctgtttttcatagcccctttgaaaatattttattgctaaatcttgtatactataccgacaattaacaggctttaaatatacataattatagccacttttaaagtaatagcttaattgatagcagtaatatgtttccaaaatctatctaaactatgctttcttggccccttttttttttctattttgactttttaaaacttctgtattcagtccctcaatctaaacaactattacctcagtagacttgcaaccaggtctttatgttcttgtaatttgaaacttttaaattttaacaactatttggaaaatcatcatctcctttatttgtttatgatgagttacttaatataaatttgtttatattttatcataatcacatatcaaaatgactttgttttattctccttttcacttaataatgtttatttcatttttttcccttactaaattaattgaaaattctaggcttttatttactgagcatagtttgatgattatatcattacatgtatccatgtttatttgtattatgaaaaattacacgaagtctgtagcatgtgtttacttttgaagtaggaagtatgggaaatgacaagtctttgcttgctttataacatgttatattaccatgtgtctaaggacatatcttcctttcgttctatcagagacagatctttctttaattctacaggcttagtcaaatgtcataacttccagtaattctggtaagtacaccaacctatccaccagttcttgatagttcacccaattttctatttttaaataaaatgatgagttaaatatatttccaaacaaataaagcatttatatatttcagtttaaacaaagcatttatttgtggtgaaaaagaaagatgatgatgtatctggaattctagctgttcttaacaacgtgggacgaaggaaactttcagtacatgggtttaattgtcgatacaaagatgggctgctcagtccttcaaggagggacttacaaaccagcttgaaactcctgcagggtctgcctcaaccacagacaacctattagaggtcacacatgtgtcagggcagctccatctggtgaatgaatgagacagagaacaaaggcctttgccattttgaccttaagagggtcacttccgcaggcaatactccataactcgttgctagcttttctgatgttttgtcaaatctgcatttttgtttgactcttccttagcctgatactgatttctgacatctctcttcacatgtagtgagtcctaataaacatcctgtaccccatactttgcttctatttccagagaacacaattggtgacatatcaaagtaggaacattctggactctatgattatagtcctaattcatatcttgaatttcaagcattagcaacttcaggaaacccaagtggttcagcatattttcactataaatcaaatatgtaaagtcgatgtacaatcactggcccttgttggaattatttagaaagtccaaagtctcttagaaagcagaaaatatatctatggatttctgcttattaatgcattcatttaaatatctgagcattattcattcactcactcactcattctttcaaacaaattttattcccagctgtatgctggctgtcttgttaagtgcagggatacagagataaatacatgatctctgtcattaaggcacctaatgatgacagagataaatataatacataatttcgaaagcacgtgtattagaattagttttagtgttttatccatttttgtttctgtttttgtttggctgctgtaacaaattaccacaagaatagtggcttaagacaacacaaatttattgcttgcaattctggagggatggaagtccaaattcattctcacccggctaaagttaacaattttgtggggctggttctttctagaggccttgagagaatctgttttcttgtcttttccaatattcagtagctgtctgcattcgttagtcaatagcctcttcctcttattcctccagtctttctttcacttcacgtctcttattacttaccctgaccctcttgccttctgctcatatggactcttgtgactacattgggcctacctagataaccaggatgatctccaaaacccaagagccttaatttaagcacagctgcaatgtcccctttaccatgaaaggcaaaacttatagattctgaagattagaacattaaccttgggggccattattcagcctaccacactacttaactatgtgggtagagacaagggcaggtacgaagtcagaatattgctgctaagagcatcctaaggtattgaggaaggtgctgaatagtaggtcagtaaactttgccaaaacatagcttttaagtagagtgttgaaagataactaaatgtttgttagtgaagggtacagcagaggtgaagtgtaccttctaggatgagagagtagcttctgccaaggcatggaaatgacaggaaatataagtattcatggaattctagctgtcagccacaagacacatacttttcttacactttatttctgaaaaatagcatgtttcttacaattaatgagtatatttaatatagtcattaattattcctaaatattttatgttttctattaaaatatgaatataatgtaatttaataaacttggtttatatgatgctaagaaaatactgagagatgagaccaaagaacaaaatatgtgtcaaactcattacaggttgtaaccatgataaggaacttttaaaccatactggcgatcacacaaagtttttgattttcttgaagaagttatttcacatagtttgattttagttttagagaaatcacattgactgcatggaagaaaagtatgggagaggagcaatagtatagacagagaaaagagatgtttgccataatctggcaaatattgaaccctgaaggaagaaggtggtaggctgttgattgagaggcggtgagacatagaatgaccaattttggtaaatgatttgttgtaggtagtgggaagataagaaaaggtacagtaatttcaacggctccctgatttgggcaattaggagaatgattgtactttttgttgagaaaagaattgagggacagatgttttttcagagaggaatgggtgaataacaatatgtatgcattgtatttagtttttgaggcatgtgtgaaatatataagtttaaatattcatcagtatttgtatataagtgtatgaagcccaggaatgggtttttagatgaagatacagattgagcaatcattcagtggtaagttaaatctaaaatgaaaagcaaacaaacaaaaactcttctatgtactgtcaacattttgacaccaaatatgtgggttttccaaccaaccaattatgacattgggtgctcagagttaacacagatcccataggttaggggctcagacccccaagactgccaacccccccatacacaattcagatgccaactgaaatttcagggtgtcatctgtgcttctgaccaactatctctaaatcaagcgtttccatgacctcctccttgggttgaataatttcataaaacaactcacagaaatcaggataaaagcttacttgctagcttatcagtttattacaaaaggatacaactcaggaatagtcggatggaagaaaatcatacggcaaggtgtccaggagaggtgtggagcttccatgccttctcccagcacctccacaggtttgccaacccggaagctctctaaattctctgaaccctgtgcttttgggtatttacagagccttaattcttagacaatattgattaaaaccattgacctttggtgaataggttcccatagtctctcttccctcccagaaggtcaggggttggggctaaaatttccaaccctttaatcacaagattggttctcctggcaaccagtcaccatcctgaggctatcctggaaccctcagctactagtcatctcattagcatacaataaaacacttattattttggagagtccaaggattttaggtattttgtgtcagataatggagacaaagaccaaatatatatacttcttaatatatcacaaaatcacaaaaccttaagcagtgacgacacaatatgaaagaatggcatgaacatttcactttgcttatttgggctgttctggggtcagctttctctcattttggaggactgtccaattttgtgtattaatatgagatgcagtgttctgcttctcaccatggtagtataacttggactaacttttagttctctgcccactgatgacctaagttttgggaatgtgacttgggctcagccaatcagttgctcccacacaaaactttaatctggagttagaggcacaaagaagaagggttagggctcatttataagaaatttgttgtgttcagtgagagcagcaccaggaccgtagtgtgagatttctaccgtggtaggggcagagtcctaaccagactgttaaatacttatttcctgacctacttttaattttttacctcttttcaaagtctgatcgtatagttggtcattaattctcaaattcttaacattattctcataaaaaccctttactgcctcatctagctatagttgacttttcttcttacaatcatgattcataaagaagacgtatagaatgaaaaaagaaataggaggagaaattagaaaaaaaaattgatggttacagataatacgaagaacctcatatttgatggttactatcagtctgagcctttatgttttccagtaagaatcttttacttttaacctaacaattgtgaattgccatacttccttcaaacaaaacagcttcacatgcaaaaaacaagcaatgatagtcttggaattttccaagctggtttgatgaaaaagtataactggcactactgattttattgcttaggaatggattaatctctttataagtttgctttcactgcccatgcatatattgacaaaagggggtgttttattcagtttaataaagaattttatatgtgcttctgtgtgtatataagtacaaaaaggcttacttaatgtatttatatttttccaattctgaaagacctttatggactataaatattgattaaagtgatattttttctcagaaaaagattgcttaactatacccataaaatggcaatttgacggagtgtatggatcttctgcaaatactgctacacaggattaattttaaaatgtaatccagctcatatatcattaaaactttttcttcaatagtaaaaaggtcccattttcacattacaaagatcaaaacacataattttcatattgttttaaatggaaatgaaagatttcctagataaaatataaagaatacaaaattggaatatgaattatttcatgtttatgaaagcatcaaaacataatatgaattttctagtttagtagatcatgttgagatttttgcccacaaataaacttttgggttatttaatacaagtatatgaacatctaatcattcctttattcacttctgccaattttattttgtgccttccatgtggcagtaactgcattaagtatagaatgatacatatatagagatttttctagtgaagggctcaaggtttgagttgatggtagaaattgattcagttataaataggtataaggcaaaatagagaatgaatgtatagtcagaaaattttaaaaaatagagtaagaaactgtaatataaaacttaaatagttaaatggattttattagtctataaagtcaaggatttgtctatttagaaaaggagcaggctataccatttatcaatattttcctactttaatttcaaaataattcacatttattagagtggatataatatattcgcacaaaggaaacttaacatgaggagaactccttaagaattataaagtattattcaatcacaaacctaaatgacctaaaacgtaaagtgcagagaaagcaaaaggcaggtgacctagaaggtaataatacttaagggaaatattctgtcagtttctaggtgcccttgaatgctcacactcttatgtttttgccgtgagcaataagctggagactctattttttgttctttctatcaaaataagactgattttctacgttataattcccattgccttagaatttcattacttgagttatagtagttaagcaagtcttttaattatagcataaaatgctctagttacaatagtttagggaatatattaatcactcactgacatttacatatttaaatgtcttgtgctccacatacaattctaaaaagagaaaagaggttttttaacatttcttaactgagttactttaaataaggtgattttaagagcagtatctgtttatatcaagcatcaaacattttggtggtgacaagttatatgaatgtatttagaataaaggttaaatgccaatttcatttttcagatttaataagtagatttcaagtataaaaaattattattgaataaagccacatattttaaaagtctacttaatatctccactgagagtctatatttaagtggagaactaaatacttagatttcacaataaacaaacaatgctttacatttgattcttaattctaatatagttaaaattaaatgtaatataaaagctgtataaatatttttaagtttacataatgaggcaaatgaaattgagtattgtgtatgcatgtaaatgtacacgtgttttggttgctgtttattttttgtttttcttcaaaatcaatagacaaatatcaaagctttcagattcattgatgtgataacatttgatatttcacttcaatgtgtaaataatttgactgcaaatttatacgtgtgttttattcaagggttttctgatttttaaaaaaatcttgctgggccacaaatattgccaggctttgtcacaattttttgtttgtttgttttgttttttgtttttgagacggagtcttgctctgttgccagactgaagtgcagtggcgtgatcttggcttactgcaacctccacctcccgggttcaagcgattctcctgcctcagccatccgagtagctgggactataggcgtgcaccaccatgcccagctaatttttgtatttttagtagagacggggtttcaccacggtggccaggatggtctcaatttcttgacctcatgattcacccgccttggcctcccaaagtgctgggattacaggtgtgaaccaccaagcccggcctgtcacaagtttttagtgttctattttaatacagaaattagataaatccaaagagaaagacatttcatatgtggtagagttgtcggaagaaatgagagtcttataaataactttaaaaattgtgaagaaataaagacaaaatagtcctatgcagtttgatttaaatatattcttaataagagctacttttgtgaaaaccagaatattgaaacatgtagatatggatcttcattagtgactgacataatatattgttattgttactattttattgtatcagccaactaatattgagtgctttgtgtatcctaagcactatgctaaacactgtaccagtattacctgatataatcatattaatatttattatttcacttttcatatgaaaaaattgaagcacagattaagacactccgaaatcatacctctattgattatcagcaccaggatttgaattgaggcactctgatccagagaagcttttgtttccatgaagcttatgttggggaaaaataatcaaattgcctgtactcagttgtataaatataggttggttgtagatgattctggctgattcaacagaaaagaaatttattcaaaggatatcacacagttttcataacagttaagaatacagaggaaacagggcaccagggtaagtacagaccaaagtccaaaaccactgccaaagttgcagcaaggagaacagcacaaatttgcttgctgtcacccgccactagatgcttttgtttggagccttgaacttgacttacactgccactgacatcagcaccagtgctctctgtgtactaggaggtggaccttgtgaccgttgctgaactcaaaagtcagatgtttctgctgtgaaatagatacctaatacagaacctgcttcctcattcattccctcccaaatcatatgcttgtagtgtggctagagtttctgtttctccttggtccaggcagaatttatgaagcttgctatttatcgccttaaagattagaagaatattcataaggtattagattgccataaggttgaacaaatcaacattcaacttcaaggattcaacattgttttgttttcttttgggatacctctgcagcagttcaaatcttatttctgcccttggacaaccaggtttataaatattgcagattctccactgactgctttgatcctatcttctatatttatgtatactaattagcatataataaaagattatgttacagaatctcaaaattagtaattatgaattgagatggtgttatacagtacactaacatccagagacttgtttattccaaggaaaatatttagagatattaaatgatatttctatcctttagacatatacattttttagcttacagcctgctttaggcaagcaacagactctcaggatctgctcctaccagggtctgaacatttcctcccagtttttaaagaaacaaattcaaataacattgtaacctccagaggaaagttcaagctcttttatagtattgtttaaacagtacagctgaggaaactaaagacagagaagttaaatgccttggcacttagtctagatttacaataaactcctctctacttaggacccactaacaggggctgcatttacaccaaaaccatgaaggtggcccaagtcatcactgagaagtagtacaagcaccgagggaatgacttcaacaggaacaagaaagcgtggaaggagatcctagcaggaagctccacaagaagatagcatgttacgtcttgcattggatgaagcaggttcagagagacctagtgacagctatctccgtcaaggtgcagaaggagagatcattgaagtagattttcatgcaaaaaaaaaaatgttgaagtctttggacttcgggagtctgtccaaactgcaggtcactcagcctacagttgggatgaatttcaaaacaccagttggagccggttgaatctttctgctatgctgtaatattttcagtaaacccagaacaacaacaacaacaaaacacaatggaggagaagcagccaagtctcttggtttacagagtagctcctaataccccttgctgtctgtctcagtgcccaatgggaagatagtcaaaacaatattcacacctgtgattcatctctctacatgcagtgtgtgtgaatctttatatactgcatattaaggatctgtctttacagataaaaactaaagcattgaaggaactccttgttttgacttatcaaagtccttaagaaaatactagaaaattatagccattgtttcaaattttagctttatattatcacttgaaatgtgatgaaatgtggctgatagataataattcactgataacctacagacaattcccatcttaaaatggaccattggattgaagaattaaataaaattgagggttttccttacatgttttgtctaaagagcgaagtagaaacaactgttcatagatcttcattgaggattcgcatgtgaagtaagtactcctaaacataaacagtgacttatcaaccagttccataaatcatgaacaaaatatttgtcccagagagactatttttccaccacatctcttgtaataaacacagagccagttcagttaaaatagtttaagggtggacggttcagggcttgctgagtggcactcagtaagaaaacccagcagaacatttacttctctctttattccagagcatcaatggccaaggctggaagatcccagaacactgaacagacatttggtctcttatggcctgccaattttcacagtgggttccaacgctttgggtcaaaccaaaatagacctgttagaaaaatgtcggttggaatacgctaacaataagacagaataaatgtgattatttggcctcatttttataggacttgagtaattttattataacattcttgagggctggaaaatctgaatgttaggacaccaaatatctccagaaaacaagttttatatttctaatcctgcataataaacctggggccactgcaggcctcattaataaaaacctaatggtataacaataatgaggaggaaatgccaatgccgcacaaatctgttgagactaaaatatttctcaccccagcaggcttggtgcatttgacacttcatgatatcagccaaagtggaactaaaaacagctcctggaagaggactatgacatcatcaggttgggagtctccagggacagcggaccctttggaaaaggactagaaagtgtgaaatctattagtcttcgatatgaaattctctgtctctgtaaaagcatttcatatttacaagacacaggcctactcctagggcagcaaaaagtggcaacaggcaagcagagggaaaagagatcatgaggcatttcagagtgcactgtcttttcatatatttctcaatgccgtatgtttggttttattttggccaagcataacaatctgctcaagaaaaaaaaatctggagaaaacaaaggtgcctttgccaatgttatgtttctttttgacaagccctgagatttctgaggggaattcacataaatgggatcaggtcattcatttacgttgtgtgcaaatatgatttaaagatacaacctttgcagagagcatgctttcctaagggtaggcacgtggaggactaagggtaaagcattcttcaagatcagttaatcaagaaaggtgctctttgcattctgaaatgcccttgttgcaaatattggttatattgattaaatttacacttaatggaaacaacctttaacttacagatgaacaaacccacaaaagcaaaaaatcaaaagccctacctatgatttcatattttctgtgtaactggattaaaggattcctgcttgcttttgggcataaatgataatggaatatttccaggtattgtttaaaatgagggcccatctacaaattcttagcaatactttggataattctaaaattcagctggacattgtctaattgttttttatatacatctttgctagaatttcaaattttaagtatgtgaatttagttaattagctgtgctgatcaattcaaaaacattactttcctaaattttagactatgaaggtcataaattcaacaaatatatctacacatacaattatagattgtttttcattataatgtcttcatcttaacagaattgtctttgtgattgtttttagaaaactgagagttttaattcataattacttgatcaaaaaattgtgggaacaatccagcattaattgtatgtgattgtttttatgtacataaggagtcttaagcttggtgccttgaagtcttttgtacttagtcccatgtttaaaattactactttatatctaaagcatttatgtttttcaattcaatttacatgatgctaattatggcaattataacaaatattaaagatttcgaaatagaatatgtgaattgttcacatacatagaaatgaaaagttcatttcgtaaagcaagatgctgggtgaaagagtgcttttgattgaaagatcactagattagtagagggcaagacttctagtccctaatctacccttaatagccatgtggtcacgtgtaagtcagtgaacccatctcattctcctcatacttttttcatctctaaaatgagggtataatttaagctcttcatttttttttttttttgagatagagttttgctcttgtcacccaggttggagtgcaatggcacgatctcagctcactgcaaccctctgcttcctcggttcaagtgattctcctgcttcagcctcccaagtagccgggattacaggtgcccgccaccacatctggctaattttttgtattttcaccatgttggccaggctggtctcgaacccctacctcaggtgatccctcgcctcggcctctcaaagtgctgggattacaggtgtgagccaccacgcccagcccaatatcagtttttcttttttaacacaaggctaacacaatcaaaatactagctaggggagaaaaaaaaaataaggcactgtttatgtgtaacaggctcttgttgcaatcactgggcagacaataaacagtaagaatcaatccttttcatatatccttcttgcagaatacataaaatcccacaaatggctatcttcctttttatgatatttggagaattgtagctaagtgacagatattttgcttgggtgtatagaccacaaaggactgtgtttgatgatggtttgcataaaattataccttagtttttactttgtatgttacatgttagatttagagtatgaaaattagtagggaggattattaacaaagaacagggcaagaggagtagaattaaacctcttctaatacctgtgcacaagtaggcttttcagaaactctacaaccctacataaactggatagttagaaaagcacactcccaaggaaggcggttatgttttgcagtttgaatcagaagaatagagctatagcaatcttcattctatagtaacattaaagagcctggtttatattatagcagtcattaagatttaaaaatttacatcttgccgttcttcttactcacagattttcgagaggtaatgtaatgatccacgaggtgagaatcactgccttttataatgcgattaaattgcatgaacaaagtttccaacaaataacagtaataaaaagaaacatgtattagcacttaataagccaggtgctgtacgacgtgtgttacatgctttcaatccatgaactggtaaactggtactagtatctctattggacatgtgaggaaaccaaatggagttgataaacagtagagttaaaaattactcttcatatattatattgcctcaatctcacagacatctctgctaccaaaagctatcatatctagatatgcggcataaggatgaccttggggcacactagaattctttgagagaattctggcagagaaaacaaatatttattcctacaataaaacccagcattttacaggttttatttttaactatgaagtattgttatctgtatctttcatataagtgtgcccggaatttatttcttctggtgggttcttggtctcgctgactccaagaatgaaaccgcagacccttgaggtgagtgtcacagttcttaaagatggtgtgttcagagtttgttccttcagatgttcagatgtgtccggagtttctcccttatggtgagttcgtggtctcgctgacttcaacaatgaagccgcagacctttgcagtgagtgtgtgacagttcttaaaggcagtgcgtccagagttgtttgttcctcccggtaggttcgtggtctcgctgatgtcaggaatgaagctgcagaccctcgcggtaagtgttacagctcataaaggtagtgcaaacccaaacagtgagcagtagcaagatttattatgaagagcaaaagaacaaagcttccccaccatagaaacggaccagaattggttgctgctgctgtggtagccagcttttattcccttatttggccacacccacatcctgctgattggcccattttacagaatgctgattggtccattttatagcgtgctgattggtgcgtttttacagagtgctgattggtgcatttacaatcctttagctagacacagagtgctgattggtgcctttataatcctttagctagacacaaaagttctacaagtccccacccaacccagaagctccgctggcttcacctctcgtaaggaaattgaggttcaaacaagtttcaaagtgctaaaactacagtttctcattctctgcaactggatttccactcatgtgtttgaatcccaggctctaagacttaacttgccattctgtgactttatgttcctgcaatttacacaaagctactatctgtcacatctctggtgttaacttcagactaaacttctttttgattcacaatgaccacacactttttggttgaggttttgctatcggtttattgtactggttaatagagagcttcttccagaaatttgagtagatggaagaggaagtagcacattcctaaaaatgtaccatgcctttcaagtcacaagcatccctatcacatggctgtcaagggtggctcagaataggtagagttaagaatttaaagtaaattggtgtaagcgatgaaagcttcatctaaaagcttatattacatcaactgaaatgtaaaataattggaacattttccaggcatccctgttatttatttgtctctctttccttgcttgcctacttcaaaagtcatatggcatggtgactagaactgtcctgccaaagagtttgtcaatataagattcctttctttgtaaacattctaccttggggcttcatttataatcaaaaggagtactgtaacctgtcaaaaaaaagctacctgtgacaatatattatgtgatggttacctgcagtaaggtggtggcaataaataaataaataatcacagaatgaaaccgagcagaactgtcagagaaatggtcagaattcacactctgaagaacacggctatacagtaataatcataataaatagccactcaatccaaaacatcactgggcgacttgtcacatatataatcagtggagatgtgattgaagcacaaggcttaagtgaatgtctagagagctaattgattcatttttatggaaattttacttattttaaatgtcatccctgaccatcttgaacttttacttgaagatttatttttttttttaaatcactgtttattagatttaggtattctggtctttgtttttcttttttatctatgtatgatttttatttttttatgcagtgtccttaagcttcatcaatgagaagaaatgtattaaaatccatttattcttaccct

SEQ ID NO: 6 - DNA, 50 bp, Homo sapiens
ctaatgaatatataaatataaagcataacaataataatacaataccaccc

SEQ ID NO: 7 - DNA, 22 bp, Homo sapiens
aacctgtgtctgcaacttcctc

SEQ ID NO: 8 - DNA, 21 bp, Homo sapiens
tcatgaggcatttcagagtgc

SEQ ID NO: 9 - DNA, 22 bp, Homo sapiens
cctgtgcacaagtaggcttttc

SEQ ID NO: 10 - DNA, 22 bp, Homo sapiens
cctcagaaatctcagggcttgt

SEQ ID NO: 11 - DNA, 22 bp, Homo sapiens
cttaggaaagcatgctctctgc

SEQ ID NO: 12 - DNA, 22 bp, Homo sapiens
ttgttggaaactttgttcatgc

SEQ ID NO: 13 - DNA, 22 bp, Homo sapiens
cggagaaatcctggttacactg

SEQ ID NO: 14 - DNA, 22 bp, Homo sapiens
taaatgcacttgccactcactc

SEQ ID NO: 15 - DNA, 22 bp, Homo sapiens
catcccttgcatgatatgtgtg

SEQ ID NO: 16 - DNA, 22 bp, Homo sapiens
ttgccttaatcatgtgccagat

(19) United States
(12) Patent Application Publication
(10) Pub. No.: US 2002/0155463 A1
(43) Pub. Date: Oct. 24, 2002

(54) Prostate polynucleotides and uses

(76) Inventors: Zairen Sun, Rockville, MD (US); Xuan Li, Silver Spring, MD (US); Gilbert Jay, North Bethesda, MD (US)

Correspondence Address: ORIGENE TECHNOLOGIES, INCORPORATED, 6 TAFT COURT, SUITE 100, ROCKVILLE, MD, 20850, US

(21) Appl. No.: 09/996,956

(22) Filed: Nov. 30, 2001

Related U.S. Application Data
(60) Provisional application No. 60/250,354, filed on Dec. 1, 2000.

(51) Int. Cl. C12Q 1/68; C07H 21/04

(52) U.S. Cl. 435/6; 536/24.3; 536/23.2

(57) ABSTRACT

The present invention relates to all facets of novel polynucleotides, such as PR33a, PR33b, and PRB008, and their applications to research, diagnosis, forensics, and therapy. The polynucleotides are selectively expressed in prostate and therefore are useful in variety of ways, including, but not limited to, as molecular markers, as drug targets, and for detecting, diagnosing, staging, monitoring, prognosticating, preventing or treating, or determining predisposition to diseases and conditions of the prostate.

This application claims the benefit of U.S. Provisional Application Serial No. 60/250,354, filed Dec. 1, 2000, which is hereby incorporated by reference in its entirety.

### DESCRIPTION OF THE DRAWINGS

SEQ ID NO. 1 is a nucleotide sequence of a cDNA for PR33a.

SEQ ID NO. 2 is a nucleotide sequence present in Pr33a, but absent from Pr33b.

SEQ ID NO. 3 is a nucleotide sequence of a cDNA for PR33b.

SEQ ID NO. 4 is a nucleotide sequence of a cDNA for Prb008.

SEQ ID NO. 5 is a genomic sequence for PR33a and PR33b.

SEQ ID NO. 6 shows a genomic promoter sequence for PR33a and PR33b.

### DESCRIPTION OF THE INVENTION

The present invention relates to all facets of novel polynucleotides, the polypeptides they encode, antibodies and specific binding partners thereto, and their applications to research, diagnosis, drug discovery, therapy, clinical medicine, forensic science and medicine, etc. The polynucleotides are differentially expressed in prostate cancer and are therefore useful in variety of ways, including, but not limited to, as molecular markers, as drug targets, and for detecting, diagnosing, staging, monitoring, prognosticating, preventing or treating, determining predisposition to, etc., diseases and conditions, especially relating to prostate cancer. The identification of specific genes, and groups of genes, expressed in pathways physiologically relevant to prostate cancer permits the definition of functional and disease pathways, and the delineation of targets in these pathways which are useful in diagnostic, therapeutic, and clinical applications. The present invention also relates to methods of using the polynucleotides and related products (proteins, antibodies, etc.) in business and computer-related methods, e.g., advertising, displaying, offering, selling, etc., such products for sale, for commercial use, for licensing, for analysis, etc.

Prostate cancer is the most common form of cancer diagnosed in the American male, occurring predominantly in males over age 50. The number of men diagnosed with prostate cancer has steadily increased as a result of the increasing population of older men. The American Cancer Society estimates that in the year 2000, about 180,000 American men were diagnosed with prostate cancer and about 32,000 died from the disease. In comparison, 1998 estimates for lung cancer in men were 171,500 cases and 160,100 deaths, and for colorectal cancer, the estimates were 131,600 cases and 56,000 deaths. Despite these high numbers, 89 percent of men diagnosed with the disease will survive at least five years and 63 percent will survive at least 10 years.

Patients having prostate cancer display a wide range of phenotypes. In some men, following detection, the tumor remains a latent histological tumor and does not become clinically significant. However, in other men, the tumor progresses rapidly, metastasizing and killing the patient in a relatively short time. Prostate cancer can be cured if the tumor is confined to a small region of the gland and is discovered at early stage. In such cases, radiation or surgical removal often results in complete elimination of the disease. Frequently, however, the prostate cancer has already spread to surrounding tissue and metastasized to remote locations. In these cases, radiation and other therapies, are less likely to effect a complete cure.

Androgen deprivation is a conventional therapy to treat prostate cancer. Androgen blockade can be achieved through several different routes. Androgen suppressive drugs include, e.g., Lupron (leuprolide acetate), Casodex (bicalutamide), Eulexin (flutamide), Nilandron (nilutamide), Zoladex (goserelin acetate implant), and Viadur (leuprolide acetate), which act through several different mechanisms. While these drugs may offer remission and tumor regression in many cases, often the therapeutic effects are only temporary. Prostate tumors lose their sensitivity to such treatments, and become androgen-independent. Thus, new therapies are clearly needed.

The first clinical symptoms of prostate cancer are typically urinary disturbances, including painful and more frequent urination. Diagnosis for prostate cancer is usually accomplished using a combination of different procedures. Since the prostate is located next to the rectum, rectal digital examination allows the prostate to be examined manually for the presence of hyperplasia and abnormal tissue masses. Usually, this is the first line of detection. If a palpable mass is observed, a blood specimen can be assayed for prostate-specific antigen (PSA). Very little PSA is present in the blood of a healthy individual, but BPH and prostate cancer can cause large amounts of PSA to be released into the blood, indicating the presence of diseased tissue. Definitive diagnosis is generally accomplished by biopsy of the prostate tissue.

No single gene or protein has been identified which is responsible for the etiology of all prostate cancers. Although PSA is widely used as a diagnostic reagent, it has limitations in its sensitivity and its ability to detect early cancers. It is estimated that approximately 20% to 30% of tumors will be missed when PSA is used alone. It is likely that diagnostic and prognostic markers for prostate cancer disease will involve the identification and use of many different genes and gene products to reflect its multifactorial origin.

Nucleic Acids

The present invention relates to polynucleotides which are selectively expressed in prostate. The polynucleotides are primarily noncoding. Only very short open reading frames are identified within them. A polynucleotide sequence of the invention can contain the complete sequence as shown in SEQ ID NO. 1, 3, and 4, degenerate sequences thereof, anti-sense, muteins thereof, and fragments thereof.

PR33a (SEQ ID NO. 1) and PR33b (SEQ ID NO. 3) are structurally related sequences. PR33a is about 5217 nucleotides in length, including a polyA tail, and has two Alu-type sequences at about nucleotide positions 319-440 (Alu I) and 2010-2226 (Alu II), both in a reverse or antisense orientation. PR33b is about 5093 nucleotides in length, including a polyA tail, and has a single Alu sequence in reverse at nucleotide positions 1837-2092 which corresponds to the Alu II sequence of PR33a, but is missing the Alu I sequence. SEQ ID NO. 2 is the nucleotide sequence which is present in Pr33a, but absent from Pr33b. PR33a has an additional CAG triplet (the Alu I sequence, itself, has a 3′ CAG triplet at its terminus) adjoining the 3′ end of its Alu I sequence which is absent from PR33b. Other than these two differences, PR33a and PR33b share the same nucleotides sequence and appear to arise from the same gene (see below). In addition to the transcripts corresponding to PR33a and PR33b, other cDNAs arising from the same gene have been detected. These are described in more detail below in the section describing genomic DNA.

On a Northern blot, two transcripts are detected, at about 5 kb and 7 kb, the former probably corresponding to PR33a and PR33b. The 7 kb transcript appears to correspond to a different splice variant. PCR analysis of 24 human tissues showed expression of PR33a and PR33b in prostate, but absent, or at very low levels, in brain, heart, kidney, spleen, liver, colon, lung, small intestine, muscle, stomach, testis, placenta, salivary gland, thyroid, adrenal gland, pancreas, ovary, uterus, skin, peripheral blood leucocytes, bone marrow, fetal brain, and fetal liver. Both polynucleotides were present in prostate cancer, but in variable amounts.

PRB008 (SEQ ID NO. 4) is about 2085 nucleotides in length, including a polyA tail. It is also present in the prostate, but absent, or at very low levels, in brain, heart, kidney, spleen, liver, colon, lung, small intestine, muscle, stomach, testis, placenta, salivary gland, thyroid, adrenal gland, pancreas, ovary, uterus, skin, peripheral blood leucocytes, bone marrow, fetal brain, and fetal liver.

By the phrase “selectively expressed,” it is meant that a nucleic acid molecule comprising the defined sequence of nucleotides, when produced as a transcript, is characteristic of the tissue or cell-type in which it is made. This can mean that the transcript is expressed only in that tissue and in no other tissue-type, or it can mean that the transcript is expressed preferentially, differentially, and more abundantly (e.g., at least 5-fold, 10-fold, etc., or more) in the prostate when compared to other tissue-types. In either case, a selectively expressed polynucleotide is a useful prostate marker and probe because its occurrence in a sample indicates the presence of prostate, having significant applications in diagnosis, therapy, and related areas. This is discussed in more detail below.

A mammalian polynucleotide, or fragment thereof, of the present invention is a polynucleotide having a nucleotide sequence obtainable from a natural source. It therefore includes naturally-occurring normal, naturally-occurring mutant, and naturally-occurring polymorphic alleles (e.g., SNPs), etc. By the term “naturally-occurring,” it is meant that the polynucleotide is obtainable from a natural source, e.g., animal tissue and cells, body fluids, tissue culture cells, forensic samples. Natural sources include, e.g., living cells obtained from tissues and whole organisms, tumors, cultured cell lines, including primary and immortalized cell lines. Naturally-occurring mutations can include deletions (e.g., a truncated amino- or carboxy-terminus), substitutions, inversions, RNA editing modifications, or additions of nucleotide sequence. These genes can be detected and isolated by polynucleotide hybridization according to methods which one skilled in the art would know, e.g., as discussed below.

A polynucleotide according to the present invention can be obtained from a variety of different sources. It can be obtained from DNA or RNA, such as polyadenylated mRNA or total RNA, e.g., isolated from tissues, cells, or whole organism. The polynucleotide can be obtained directly from DNA or RNA, or from a cDNA library. The polynucleotide can be obtained from a cell or tissue (e.g., from an embryonic or adult tissue) at a particular stage of development, having a desired genotype, phenotype etc.

The following polynucleotides (incorporated by reference in their entirety by reference to the Genbank Accession number and that disclosed in P.N.A.S., 97:12216-12221, 2000, the antisense of these polynucleotides, the regions thereof which overlap with PRB008, and fragments thereof, can be excluded from the present invention:

| | | Score | E |
|---|---|---|---|
| | | (bits) | Value |
| gb\|BE973585.1\|BE973585 | 601680950F1 NIH_MGC_83 Homo sapiens . . . | 1170 | 0.0 |
| gb\|BE673278.1\|BE673278 | 7d32d09.x1 NCI_CGAP_Pr28 Homo sapien . . . | 942 | 0.0 |
| gb\|AI420333.1\|AI420333 | te93b01.x1 NCI_CGAP_Pr28 Homo sapien . . . | 930 | 0.0 |
| gb\|AI732058.1\|AI732058 | nc19c11.x5 NCI_CGAP_Pr1 Homo sapiens . . . | 870 | 0.0 |
| gb\|AI820968.1\|AI820968 | nc19c11.y5 NCI_CGAP_Pr1 Homo sapiens . . . | 854 | 0.0 |
| gb\|AA226269.1\|AA226269 | nc19c11.r1 NCI_CGAP_Pr1 Homo sapiens . . . | 852 | 0.0 |
| gb\|BE673322.1\|BE673322 | 7d33b09.x1 NCI_CGAP_Pr28 Homo sapien . . . | 821 | 0.0 |
| gb\|AA216357.1\|AA216357 | nc16a06.s1 NCI_CGAP_Pr1 Homo sapiens . . . | 785 | 0.0 |
| gb\|AA630840.1\|AA630840 | nt57e01.s1 NCI_CGAP_Pr3 Homo sapiens . . . | 728 | 0.0 |
| gb\|AA228731.1\|AA228731 | nc46c10.r1 NCI_CGAP_Pr3 Homo sapiens . . . | 670 | 0.0 |
| gb\|AI869019.1\|AI869019 | wc18a06.x1 NCI_CGAP_Pr28 Homo sapien . . . | 664 | 0.0 |
| gb\|AW445020.1\|AW445020 | UI-H-BI3-aka-g-01-0-UI.s1 NCI_CGAP_S . . . | 660 | 0.0 |
| gb\|AA229424.1\|AA229424 | nc46c10.s1 NCI_CGAP_Pr3 Homo sapiens . . . | 660 | 0.0 |
| gb\|AA226583.1\|AA226583 | nc19c11.s1 NCI_CGAP_Pr1 Homo sapiens . . . | 650 | 0.0 |
| gb\|AA229229.1\|AA229229 | nc45d06.s1 NCI_CGAP_Pr3 Homo sapiens . . . | 620 | e-175 |
| gb\|BE673425.1\|BE673425 | 7d35a06.x1 NCI_CGAP_Pr28 Homo sapien . . . | 611 | e−172 |
| gb\|AA639902.1\|AA639902 | np08f05.s1 NCI_CGAP_Pr3 Homo sapiens . . . | 599 | e−169 |
| gb\|AA579452.1\|AA579452 | nf29f09.s1 NCI_CGAP_Pr1 Homo sapiens . . . | 595 | e−167 |
| gb\|AI918896.1\|AI918896 | tu13d02.x1 NCI_CGAP_Pr28 Homo sapien . . . | 557 | e−156 |
| gb\|AA228669.1\|AA228669 | nc16a06.r1 NCI_CGAP_Pr1 Homo sapiens . . . | 553 | e−155 |
| gb\|AI810789.1\|AI810789 | tu21b03.x1 NCI_CGAP_Pr28 Homo sapien . . . | 539 | e−151 |
| gb\|AI927859.1\|AI927859 | wo66b03.x1 NCI_CGAP_Pr22 Homo sapien . . . | 525 | e−146 |
| gb\|AI971034.1\|AI971034 | wr23b04.x1 NCI_CGAP_Pr28 Homo sapien . . . | 521 | e−145 |
| gb\|AA688095.1\|AA688095 | nv14g08.s1 NCI_CGAP_Pr22 Homo sapien . . . | 500 | e−139 |
| gb\|AI820962.1\|AI820962 | nc13d11.y5 NCI_CGAP_Pr1 Homo sapiens . . . | 448 | e−123 |
| gb\|AI732150.1\|AI732150 | nc13d11.x5 NCI_CGAP_Pr1 Homo sapiens . . . | 448 | e−123 |
| gb\|AA229455.1\|AA229455 | nc45d06.r1 NCI_CGAP_Pr3 Homo sapiens . . . | 448 | e−123 |
| gb\|AA230302.1\|AA230302 | nc13d11.r1 NCI_CGAP_Pr1 Homo sapiens . . . | 440 | e−121 |
| gb\|AA230247.1\|AA230247 | nc13d11.s1 NCI_CGAP_Pr1 Homo sapiens . . . | 400 | e−109 |
| gb\|AA229408.1\|AA229408 | nc47f03.r1 NCI_CGAP_Pr3 Homo sapiens . . . | 394 | e−107 |
| gb\|AA229263.1\|AA229263 | nc47f03.s1 NCI_CGAP_Pr3 Homo sapiens . . . | 254 | 6e−65 |
| gb\|AI804949.1\|AI804949 | tu43g09.x1 NCI_CGAP_Pr28 Homo sapien . . . | 182 | 2e−43 |
| gb\|AI202408.1\|AI202408 | qs71d10.x1 NCI_CGAP_Pr28 Homo sapien . . . | 182 | 2e−43 |

Genomic

The present invention also relates genomic DNA from which the polynucleotides of the present invention can be derived. A genomic DNA coding for a human, mouse, or other mammalian polynucleotide, can be obtained routinely, for example, by screening a genomic library (e.g., a YAC library) with a polynucleotide of the present invention. A genomic sequence for PR33a and PR33b is shown in SEQ ID NO. 5. The gene has exons at about nucleotide positions 1923-2156 (exon 1), 2917-3000 (exon 2), and 5852-5973 (exon 3 which corresponds to Alu I). In the 3′ region of the gene downstream from exon 3, there appear to be multiple splice acceptor sites, giving rise to at least four different exons, beginning at about nucleotide positions 24,576, 26,196, 27,706, and 27,709. Multiple alternatively-spliced transcripts are detected, including those already described, PR33a comprising, from 5′ to 3′, exon 1, exon 2, exon 3, and an exon from 27,706-32,463; and PR33b comprising, from 5′ to 3′, exon 1, exon 2, and an exon from 27,709-32,463. Additional transcripts include, e.g., PR33b-12 comprising exon 1, exon 2, and an exon from 27,706-32,463; and PR33-2 comprising exon 1, exon 2, exon 3, and an exon from 27,709-32,463. Other alternatively-spliced transcripts arising from different combinations of the mentioned exons are included in the present invention, including, e.g., transcripts containing the 3′ exon from 24,576-32,463, or, 26,196-32,463.

Promoter and other regulatory regions can be identified upstream of coding and expressed RNAs, and assayed routinely for activity, e.g., by joining to a reporter gene (e.g., CAT, GFP, alkaline phosphatase, luciferase, galactosidase). A promoter obtained from a prostate-selective gene can be used, e.g., in gene therapy to obtain tissue-specific expression of a heterologous gene, e.g., to deliver therapeutic agents, cytotoxic agents, etc., in the treatment of prostate cancer. A promoter sequence is found at about nucleotide position numbers 509-558 as shown in SEQ ID NO. 6. The promoter, and upstream and downstream regions, can also be used as a probe to identify binding-partners which interact with it, e.g., transcription factors, regulatory factors.

The present invention relates to each of the mentioned fragments alone, or in combination with other polynucleotide fragments, e.g., as reporter genes, transcriptional signals, translational signals, enhancers, silencers, etc. The introns (e.g., 2157-2916; 3001-5851; 5974-24,575, 26,195, 27,705, or 27,708; 3001-24,575, 26,195, 27,705, or 27,708) and exons (see above) can be used as probes, as transcription and translation regulatory sequences, etc.

Constructs

A polynucleotide of the present invention can comprise additional polynucleotide sequences, e.g., sequences to enhance expression, detection, uptake, cataloging, tagging, etc. A polynucleotide can include additional non-naturally occurring or heterologous sequences, including coding sequences (e.g., sequences coding for reporter elements, antibiotic resistance, and other functional or diagnostic peptides); non-coding sequences (e.g., untranslated sequences at either a 5′ or 3′ end), or dispersed in the sequence (e.g., introns).

A polynucleotide according to the present invention also can comprise an expression control sequence operably linked to it. The phrase “expression control sequence” means a polynucleotide sequence that regulates transcription (and, optionally translation if a coding sequence is attached) of a polynucleotide to which it is functionally (“operably”) linked. Expression control sequence includes, e.g., promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the polynucleotide. For example, when a promoter is operably linked 5′ to a coding sequence, the promoter drives transcription of the polynucleotide sequence. Expression control sequences can be heterologous or endogenous to the normal gene. The expression control sequences can be of any type, e.g., constitutive, inducible, tissue-specific, etc. An inducible expression control sequence can respond to endogenous or exogenous signals.

A polynucleotide of the present invention can also comprise nucleic acid vector sequences, e.g., for cloning, expression, amplification, selection, etc. Any effective vector can be used. A vector is, e.g., a polynucleotide molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60, pQE-9 (Qiagen), pBS, pD10, Phagescript, phiX174, pBK Phagemid, pNH8A, pNH16a, pNH18Z, pNH46A (Stratagene); Bluescript KS+II (Stratagene); ptrc99a, pKK223-3, pKK233-3, pDR540, pRIT5 (Pharmacia). Eukaryotic: PWLNEO, pSV2CAT, pOG44, pXT1, pSG (Stratagene), pSVK3, PBPV, PMSG, pSVL (Pharmacia), pCR2.1/TOPO, pCRII/TOPO, pCR4/TOPO, pTrcHisB, pCMV6-XL4, etc. However, any other vector, e.g., plasmids, viruses, or parts thereof, may be used as long as they are replicable and viable in the desired host. The vector can also comprise sequences which enable it to replicate in the host whose genome is to be modified.

Hybridization

A polynucleotide in accordance with the present invention can be selected on the basis of polynucleotide hybridization. The ability of two single-stranded polynucleotide preparations to hybridize together is a measure of their nucleotide sequence complementarity, e.g., base-pairing between nucleotides, such as A-T, G-C, etc. The invention thus also relates to polynucleotides, and their complements, which hybridize to a polynucleotide comprising a nucleotide sequence as set forth in SEQ ID NOS. 1, 3, 4, and genomic sequences thereof. A nucleotide sequence hybridizing to the latter sequence will have a complementary polynucleotide strand, or act as a template for one in the presence of a polymerase (i.e., an appropriate polynucleotide synthesizing enzyme). The present invention includes both strands of polynucleotide, e.g., a sense strand and an anti-sense strand.

Hybridization conditions can be chosen to select polynucleotides which have a desired amount of nucleotide complementarity with the nucleotide sequences set forth in SEQ ID NOS. 1, 3, 4, and genomic sequences thereof. A polynucleotide capable of hybridizing to such sequence, preferably, possesses, e.g., about 70%, 75%, 80%, 85%, 87%, 90%, 92%, 95%, 97%, 99%, or 100% complementarity, between the sequences. The present invention particularly relates to polynucleotide sequences which hybridize to the nucleotide sequences set forth in SEQ ID NOS. 1, 3, 4, or genomic sequences thereof, under low or high stringency conditions.

Polynucleotides which hybridize to polynucleotides of the present invention can be selected in various ways. Filter-type blots (i.e., matrices containing polynucleotide, such as nitrocellulose), glass chips, and other matrices and substrates comprising polynucleotides (short or long) of interest, can be incubated in a prehybridization solution (e.g., 6×SSC, 0.5% SDS, 100 μg/ml denatured salmon sperm DNA, 5×Denhardt's solution, and 50% formamide), at 22-68° C., overnight, and then hybridized with a detectable polynucleotide probe under conditions appropriate to achieve the desired stringency. In general, when high homology is desired, a high temperature can be used (e.g., 65° C.). As the homology drops, lower washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, formamide can be omitted. See, e.g., Current Protocols in Molecular Biology, Chapter 6, Screening of Recombinant Libraries; Sambrook et al., Molecular Cloning, 1989, Chapter 9.

For instance, high stringency conditions can be achieved by incubating the blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity.

Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5+16.6 log₁₀[Na⁺]+0.41(% GC)−600/N where [Na⁺] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.

High stringency conditions can be selected to isolate sequences, and their complements, which have, e.g., at least about 90%, 95%, 97%, or 99% nucleotide complementarity between the probe (e.g., a short polynucleotide of SEQ ID NOS. 1, 3, 4, or genomic sequences thereof) and a target polynucleotide.

Hybridization, as discussed above and below, is useful in a variety of applications, including, in gene detection methods, for identifying mutations, for making mutations, to identify homologs in the same and different species, to identify related members of the same gene family, etc.

Alignments

Alignments can be accomplished by using any effective algorithm. For pairwise alignments of DNA sequences, the methods described by Wilbur-Lipman (e.g., Wilbur and Lipman, Proc. Natl. Acad. Sci., 80:726-730, 1983) or Martinez/Needleman-Wunsch (e.g., Martinez, Nucleic Acid Res., 11:4629-4634, 1983) can be used. For instance, if the Martinez/Needleman-Wunsch DNA alignment is applied, the minimum match can be set at 9, gap penalty at 1.10, and gap length penalty at 0.33. The results can be calculated as a similarity index, equal to the sum of the matching residues divided by the sum of all residues and gap characters, and then multiplied by 100 to express as a percent. Similarity index for related genes at the nucleotide level in accordance with the present invention can be greater than 70%, 80%, 85%, 90%, 95%, 99%, or more. Pairs of protein sequences can be aligned by the Lipman-Pearson method (e.g., Lipman and Pearson, Science, 227:1435-1441, 1985) with k-tuple set at 2, gap penalty set at 4, and gap length penalty set at 12. Results can be expressed as percent similarity index, where related genes at the amino acid level in accordance with the present invention can be greater than 65%, 70%, 75%, 80%, 85%, 90%, 95%, 99%, or more. Various commercial and free sources of alignment programs are available, e.g., MegAlign by DNA Star, BLAST (National Center for Biotechnology Information), etc.

Nucleic Acid Detection Methods

Another aspect of the present invention relates to methods and processes for detecting prostate in a sample using a polynucleotide in accordance with the present invention. Such a polynucleotide can also be referred to as a “probe.” The term “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence and/or absence, or it can be quantitative, e.g., in assessing amounts of a gene or gene transcript present in a sample. Probes can be useful in a variety of ways, such as for diagnostic purposes, to identify homologs, and to detect, quantitate, or isolate a polynucleotide of the present invention in a test sample.

Assays can be utilized which permit quantification and/or presence/absence detection of a target nucleic acid in a sample. Any suitable assay format can be used, including, but not limited to, e.g., Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”)(e.g., Schaefer in Gene Cloning and Analysis: Current Innovations, Pages 99-115, 1997), ligase chain reaction (“LCR”)(EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; WO97/18454; Prashar and Weissman, Proc. Natl. Acad. Sci., 93:659-663, and U.S. Pat. No. 712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. No. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). These and other methods can be carried out conventionally, e.g., as described in the mentioned publications.

Many of such methods may require that the polynucleotide is labeled, or comprises a particular nucleotide type. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection.

Detection can be desirable for a variety of different purposes, including research, diagnostic, and forensic. For diagnostic purposes, it may be desirable to identify the presence or quantity of a polynucleotide sequence in a sample, where the sample is obtained from tissues, cells, body fluids, etc. In a preferred method as described in more detail below, the present invention relates to a method of detecting a polynucleotide comprising, contacting a target polynucleotide in a test sample with a polynucleotide probe under conditions effective to achieve hybridization between the target and probe; and detecting hybridization.

Any test sample in which it is desired to identify a polynucleotide can be used, including, e.g., blood, urine, saliva, stool, swabs comprising tissue, biopsied tissue, tissue sections, cultured cells, stem cells, etc. Tissues can be of any type or stage, e.g., normal, benign, cancer, abnormal, suspect, etc.

Detection can be accomplished in combination with polynucleotide probes for other genes, e.g., genes which are selectively expressed in other tissues and cells, such as brain, heart, kidney, spleen, thymus, liver, stomach, small intestine, colon, muscle, lung, testis, placenta, pituitary, thyroid, skin, adrenal gland, pancreas, salivary gland, uterus, ovary, prostate gland, peripheral blood cells (T-cells, lymphocytes, etc.), embryo, breast, fat, adult and embryonic stem cells, specific cell-types, such as neurons, fibroblasts, myocytes, mesenchymal cells, etc.

Polynucleotides can also be used to test for mutations, e.g., using mismatch DNA repair technology as described in U.S. Pat. Nos. 5,683,877; 5,656,430; Wu et al., Proc. Natl. Acad. Sci., 89:8779-8783, 1992.

Specific Probes

A polynucleotide of the present invention can comprise any continuous nucleotide sequence of SEQ ID NOS. 1, 3, 4, or genomic sequences thereof, or a complement thereto. These polynucleotides can be of any desired size, e.g., about 7-200 nucleotides, 8-100, 7-50, 10-25, 14-16, at least about 8, at least about 10, at least about 15, at least about 25, etc. The polynucleotides can have non-naturally-occurring nucleotides, e.g., inosine, AZT, 3TC, etc. The polynucleotides can have 100% sequence identity or complementarity to a sequence of SEQ ID NOS. 1, 3, 4, or genomic sequences thereof, or it can have mismatches or nucleotide substitutions, e.g., 1, 2, 3, 4, or 5 substitutions. A specific polynucleotide sequence can also be fused in-frame, at either its 5′ or 3′ end, to various nucleotide sequences as mentioned throughout the patent, including coding sequences for enzymes, detectable markers, GFP, etc, expression control sequences, etc.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids selective for prostate.

Another aspect of the present invention is a nucleotide sequence that is specific to, or for, a selective polynucleotide. The phrase “specific sequence” to, or for, a polynucleotide, has a functional meaning that the polynucleotide can be used to identify the presence of a gene in a sample. It is specific in the sense that it can be used to detect polynucleotides above background noise (“non-specific binding”). A specific sequence is a defined order of nucleotides which occurs in the polynucleotide, e.g., in the nucleotide sequences of SEQ ID NOS. 1, 3, 4, or genomic sequences thereof, but usually rarely or infrequently in other polynucleotides, preferably not in a mammalian polynucleotide, such as human, rat, mouse, etc. Preferred polynucleotide probes of the present invention include, e.g., those shown below as SEQ ID NOS: 7-16. These include both sense and anti-sense orientations. In PCR-based methods, a pair of primers are typically used, one having a sense (forward) sequence and the other having an antisense (reverse) sequence. Specific polynucleotide probes include, for example:

| | Position* | Sequence | |
|---|---|---|---|
| PR33Fa (forward) | 205-226 | AACCTGTGTCTGCAACTTCCTC | (SEQ ID NO 7) |
| PR33F (forward) | 697-717 | TCATGAGGCATTTCAGAGTGC | (SEQ ID NO 8) |
| PR33Fb (forward) | 2678-2699 | CCTGTGCACAAGTAGGCTTTTC | (SEQ ID NO 9) |
| PR33R (reverse) | 845-866 | CCTCAGAAATCTCAGGGCTTGT | (SEQ ID NO 10) |
| PR33Ra (reverse) | 938-959 | CTTAGGAAAGCATGCTCTCTGC | (SEQ ID NO 11) |
| PR33J5R (reverse) | 2963-2984 | TTGTTGGAAACTTTGTTCATGC | (SEQ ID NO 12) |
| | Position⁺ | Sequence | |
| 205757F (forward) | 227-248 | CGGAGAAATCCTGGTTACACTG | (SEQ ID NO 13) |
| 205757R (reverse) | 653-674 | TAAATGCACTTGCCACTCACTC | (SEQ ID NO 14) |
| PRB008-3F (forward) | 1625-1646 | CATCCCTTGCATGATATGTGTG | (SEQ ID NO 15) |
| PRB008-3R (reverse) | 1939-1960 | TTGCCTTAATCATGTGCCAGAT | (SEQ ID NO 16) |

*The nucleotide position numbering for all polynucleotides is with respect to the sequence of PR33a as shown in SEQ ID NO 1.
⁺The nucleotide position numbering for all polynucleotides is with respect to the sequence of PRB008 as shown in SEQ ID NO 4.

These sequences can be used as probes in any of the methods described herein or incorporated by reference. Both sense and antisense nucleotide sequences are included. A specific polynucleotide according to the present invention can be determined routinely. A polynucleotide comprising such a specific sequence can be used as a hybridization probe to identify the presence of, e.g., human or mouse polynucleotide, in a sample comprising a mixture of polynucleotides, e.g., on a Northern blot. Hybridization can be performed under high stringent conditions (see, above) to select polynucleotides (and their complements which can contain the coding sequence) having at least 95% identity (i.e., complementarity) to the probe, but less stringent conditions can also be used. Less than sequence identity (e.g., 95%, 97%, 99% or greater) may be desired, e.g., to detect polymorphisms in the target gene. For example, SEQ ID NOS. 1, 3, and 4 represent specific alleles of PR33a, PR33b, and PRB008, but other alleles may be present in the human population pool, so the detection, diagnostic, and other methods of the present invention can be performed to identify such alleles.

A polynucleotide probe, especially one which is specific to a polynucleotide of the present invention, can be used in gene detection and hybridization methods as already described. In one embodiment, a specific polynucleotide probe can be used to detect whether a particular tissue or cell-type is present in a target sample. To carry out such a method, a selective polynucleotide can be chosen which is characteristic of the desired target tissue. Such polynucleotide is preferably chosen so that it is expressed or displayed in the target tissue, but not in other tissues which are present in the sample. For instance, if detection of prostate in a blood sample is desired, it may not matter whether the selective polynucleotide is expressed in other tissues, as long as it is not expressed in cells normally present in blood, e.g., peripheral blood mononuclear cells. Starting from the selective polynucleotide, a specific polynucleotide probe can be designed which hybridizes (if hybridization is the basis of the assay) under the hybridization conditions to the selective polynucleotide, whereby the presence of the selective polynucleotide can be determined.

Probes which are specific for polynucleotides of the present invention can also be prepared using involve transcription-based systems, e.g., incorporating an RNA polymerase promoter into a selective polynucleotide of the present invention, and then transcribing anti-sense RNA using the polynucleotide as a template. See, e.g., U.S. Pat. No. 5,545,522.

Along these lines, the present invention relates to methods of detecting prostate tissue in a sample comprising nucleic acid, comprising one or more the following steps in any effective order, e.g., contacting said sample with a polynucleotide probe under conditions effective for said probe to hybridize specifically to nucleic acid in said sample, and detecting the presence or absence of probe hybridized to nucleic acid in said sample, wherein said probe is a polynucleotide which is PR33a, PR33b, PRB008, genomic fragments thereof, complements thereof, specific fragments thereof, or a polynucleotide having about 70% or more (e.g., 80%, 90%, 95%, 99%, etc.) sequence identity thereto, or effective fragments thereof, and said polynucleotide is selectively expressed in said prostate. Contacting the sample with probe can be carried out by any effective means in any effective environment. It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa.

Generally, as used herein, the term “effective conditions” means, e.g., a milieu in which the desired effect is achieved. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

The phrase “hybridize specifically” indicates that the hybridization between single-stranded polynucleotides is based on nucleotide sequence complementarity. The effective conditions are selected such that the probe hybridizes to a preselected and/or definite target nucleic acid in the sample. For instance, if detection of a gene set forth in SEQ ID NOS. 1, 3, 4, or genomic sequences thereof, is desired, a probe can be selected which can hybridize to such target gene under high stringent conditions, without significant hybridization to other genes in the sample. To detect homologs of a gene set forth in SEQ ID NOS. 1, 3, or 4, the effective hybridization conditions can be less stringent, and/or the probe can comprise codon degeneracy, such that a homolog is detected in the sample.

As already mentioned, the method can be carried out by any effective process, e.g., by Northern blot analysis, polymerase chain reaction (PCR), reverse transcriptase PCR, RACE PCR, in situ hybridization, etc., as indicated above. When PCR based techniques are used, two or more probes are generally used. One probe can be specific for a defined sequence which is characteristic of a selective polynucleotide (e.g., SEQ ID NOS 7-16), but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

Polynucleotide Composition

A polynucleotide according to the present invention can comprise, e.g., DNA, RNA, synthetic polynucleotide, peptide polynucleotide, modified nucleotides, and mixtures thereof. A polynucleotide can be double- or single-stranded, e.g., dsDNA, DNA:RNA, triplex, etc. Nucleotides comprising a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, 8-oxo-guanine.

Various modifications can be made to the polynucleotides, such as attaching detectable markers (avidin, biotin, radioactive elements, fluorescent tags and dyes, energy transfer labels, energy-emitting labels, binding partners, etc.) or moieties which improve hybridization, detection, and/or stability. The polynucleotides can also be attached to solid supports, e.g., nitrocellulose, magnetic or paramagnetic microspheres (e.g., as described in U.S. Pat. Nos. 5,411,863; 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967; 5,476,925; 5,478,893.

Polynucleotide according to the present invention can be labeled according to any desired method. The polynucleotide can be labeled using radioactive tracers such as ³²P, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

Mutagenesis

Mutated polynucleotide sequences of the present invention are useful for various purposes, e.g., to identify functional regions and domains of the polynucleotides, to identify functional regions of genomic DNA (e.g., regulatory regions upstream of the start of transcription), to produce probes for screening libraries, etc. Mutagenesis can be carried out routinely according to any effective method, e.g., oligonucleotide-directed (Smith, M., Ann. Rev. Genet. 19:423-463, 1985), degenerate oligonucleotide-directed (Hill et al., Method Enzymology, 155:558-568, 1987), region-specific (Myers et al., Science, 229:242-246, 1985), linker-scanning (McKnight and Kingsbury, Science, 217:316-324, 1982), directed using PCR, etc. Desired sequences can also be produced by the assembly of target sequences using mutually priming oligonucleotides (Uhlmann, Gene, 71:29-40, 1988).

Polynucleotide Expression

A polynucleotide according to the present invention can be expressed in a variety of different systems, in vitro and in vivo, according to the desired purpose. For example, a polynucleotide can be inserted into an expression vector, introduced into a desired host, and cultured under conditions effective to achieve transcription of the selective polynucleotide. Effective conditions include any culture conditions which are suitable for achieving transcription, etc, of the polynucleotide, including appropriate temperatures, pH, medium, additives to the media in which the host cell is cultured (e.g., additives which amplify or induce expression such as butyrate, cycloheximide, cell densities, culture dishes, etc. A polynucleotide can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a polynucleotide of the present invention has been introduced is a transformed host cell. The polynucleotide can be extrachromosomal or integrated into a chromosome(s) of the host cell. It can be stable or transient. An expression vector is selected for its compatibility with the host cell. Host cells include, mammalian cells, e.g., COS, CV1, BHK, CHO, HeLa, LTK, NIH 3T3, 293, mammalian prostate and prostate-related cells lines, such as human PC-3 (CRL-1435), LNCaP (CRL-1740), CA-HPV-10 (CRL-2220), PZ-HPV-7 (CRL-2221), MDA-PCa 2b (CRL-2422), 22Rv1(CRL2505), NCI-H660 (CRL-5813), HS 804.Sk (CRL-7535), LNCaP-FGF (CRL-10995), RWPE-1 (CRL-11609), RWPE-2 (CRL-11610), PWR-1E (CRL 11611), rat MAT-Ly-LuB-2 (CRL-2376), etc., insect cells, such as Sf9 (S. frugipeda) and Drosophila, bacteria, such as E. coli, Streptococcus, bacillus, yeast, such as Sacharomyces, S. cerevisiae, fungal cells, plant cells, embryonic or adult stem cells (e.g., mammalian, such as mouse or human).

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, promoters of other genes in the cell signal transduction pathway, MMTV, SV40, trp, lac, tac, or T7 promoters for bacterial hosts; or alpha factor, alcohol oxidase, or PGH promoters for yeast. RNA promoters can be used to produced RNA transcripts, such as T7 or SP6. See, e.g., Melton et al., Polynucleotide Res., 12(18):7035-7056, 1984; Dunn and Studier. J. Mol. Bio., 166:477-435, 1984; U.S. Pat. No. 5,891,636; Studier et al., Gene Expression Technology, Methods in Enizymology, 85:60-89, 1987.

When a polynucleotide is expressed as a heterologous gene in a transfected cell line, the gene is introduced into a cell as described above, under effective conditions in which the gene is expressed. The term “heterologous” means that the gene has been introduced into the cell line by the “hand-of-man.” Introduction of a gene into a cell line is discussed above. The transfected (or transformed) cell expressing the gene can be lysed or the cell line can be used intact.

Antisense

Antisense polynucleotide can also be prepared from a polynucleotide according to the present invention, preferably an anti-sense to a sequence of SEQ ID NOS. 1, 3, 4, or genomic sequences thereof . Antisense polynucleotide can be used in various ways, such as to regulate or modulate expression a selective polynucleotide of the present invention, for therapeutic purposes, for in situ hybridization, etc. These polynucleotides can be used analogously to U.S. Pat. No. 5,576,208. An anti-sense polynucleotides can be operably linked to an expression control sequence. A total length of about 35 bp can be used in cell culture with cationic liposomes to facilitate cellular uptake, but for in vivo use, shorter oligonucleotides can be administered, e.g. 25 nucleotides.

Specific-binding Partners

The present invention also relates to specific-binding partners, such as polypeptides, polynucleotides, apatamers, etc., that specifically recognize a selective polynucleotide of the present invention. A specific-binding partner is a molecule, which through chemical or physical forces, selectively binds or attaches to a polynucleotide or polypeptide. Specific binding partners generally are referred to in pairs, e.g., antigen and antibody, ligand and receptor. A specific-binding partner specific for a polynucleotide means that the specific-binding partner recognizes a defined sequence of nucleotides in a polynucleotide, e.g., the sequence of SEQ ID NOS. 1, 3, 4, or genomic sequences thereof. Binding partners can be made conventionally.

The present invention thus relates to methods of detecting prostate tissue in a sample, comprising one or more of the following steps in any effective order, e.g., contacting said sample with a specific-binding partner, which is specific for PR33a, PR33b, PRB008, genomic fragments thereof, complements thereof, specific fragments thereof, or a polynucleotide having about 70% or more (e.g., 80%, 90%, 95%, 99%, etc.) under conditions effective for said specific-binding partner to specifically-bind to said polynucleotide, wherein said polynucleotide is selectively expressed in said prostate, and detecting the presence or absence of specific binding partner specifically-bound to said polynucleotide in said sample.

As mentioned for nucleic acid-based assays, the method can be accomplished in any effective format, including in solid, liquid, tissue sections, glass slides, etc., matrices, using any effective processes and means of detection as described above.

Specific-binding partners can also be used in methods of in vivo imaging using, e.g., MRI, SPECT, planar scintillation imaging. The phrase “in vivo imaging” refers to any method which allows the detection of a specific-binding partner located in a subject's body. Radionuclides, paramagnetic isotopes can be utilized. A radionuclide can be bound to a specific-binding partner either directly or indirectly using a functional group. Intermediary functional groups include, e.g., EDTA and DPTA. Examples of suitable metallic ions include, 99-Tc, 123-I, 131-I, 111-In, 97-Ru, 67-Cu, 67-Ga, 125-I, 68-Ga, 72-As, 89-Zr, 201-T1. Elements useful in MRI include, 157-Gd, 55-Mn, 162-Dy, 52-Cr, 56-Fe.

Specific-binding partners can also be isolated from natural sources. Many polypeptide and polynucleotides interact with other molecules that are found naturally in cells and tissues. Such interactions can be involved in regulating or modulating activity, e.g., as transcription factors, protein regulatory subunits, etc. Various methods can be utilized to isolated specific-binding partners, e.g., mobility shift DNA binding assays, methylation and uracil interference assays, DNAse I footprint analysis, UV cross-linking, interaction trap/two-hybrid system, affinity purification of proteins binding to GST fusions (Blanar and Rutter, Science, 256:1014-1018, 1992), phage-based expression cloning, gel band-shift assays, etc. See, e.g., U.S. Pat. Nos. 5,888,981 and 6,010,849 for gel band-shift assays and filter-binding assays.

Database

The present invention also relates to electronic forms of polynucleotides, polypeptides, etc., of the present invention, including computer-readable medium (e.g., magnetic, optical, etc., stored in any suitable format, such as flat files or hierarchical files) which comprise such sequences, or fragments thereof, e-commerce-related means, etc. Along these lines, the present invention relates to methods of retrieving prostate-specific gene sequences from a computer-readable medium, comprising, one or more of the following steps in any effective order, e.g., selecting a gene expression profile, e.g., a profile that specifies that said gene is selectively expressed in prostate, and retrieving prostate-specific gene sequences, where the gene sequences consist of PR33a, PR33b, and PRB008.

A “gene expression profile” means the list of tissues, cells, etc., in which a defined gene is expressed (i.e, transcribed and/or translated). The profile can be a list of the tissues in which the gene is expressed, but can include additional information as well, including level of expression (e.g., a quantity as compared or normalized to a control gene), and information on temporal (e.g., at what point in the cell-cycle or developmental program) and spatial expression. By the phrase “selecting a gene expression profile,” it is meant that a user decides what type of gene expression pattern he is interested in retrieving, e.g., he may require that the gene is selectively expressed in a tissue, or he may require that the gene is not expressed in blood, but must be expressed in prostate. Any pattern of expression preferences may be selected. The selecting can be performed by any effective method. In general, “selecting” refers to the process in which a user forms a query that is used to search a database of gene expression profiles. The step of retrieving involves searching for results in a database that correspond to the query set forth in the selecting step. Any suitable algorithm can be utilized to perform the search query, including algorithms that look for matches, or that perform optimization between query and data. The database is information that has been stored in an appropriate storage medium, having a suitable computer-readable format. Once results are retrieved, they can be displayed in any suitable format, such as HTML.

For instance, the user may be interested in identifying genes that are selectively expressed in prostate. He may not care whether small amounts of expression occur in other tissues, as long as such genes are not expressed in peripheral blood lymphocytes. A query is formed by the user to retrieve the set of genes from the database having the desired gene expression profile. Once the query is inputted into the system, a search algorithm is used to interrogate the database, and retrieve results.

Transgenic Animals

The present invention also relates to transgenic animals comprising differentially-regulated genes of the present invention. Such genes, as discussed in more detail below, include, but are not limited to, functionally-disrupted genes, mutated genes, ectopically or selectively-expressed genes, inducible or regulatable genes, etc. These transgenic animals can be produced according to any suitable technique or method, including homologous recombination, mutagenesis (e.g., ENU, Rathkolb et al., Exp. Physiol., 85(6):635-644, 2000), and the tetracycline-regulated gene expression system (e.g., U.S. Pat. No. 6,242,667). The term “gene” as used herein includes any part of a gene, i.e., regulatory sequences, promoters, enhancers, exons, introns, coding sequences, etc. The nucleic acid present in the construct or transgene can be naturally-occurring wild-type, polymorphic, or mutated.

Along these lines, polynucleotides of the present invention can be used to create transgenic animals, e.g. a non-human animal, comprising at least one cell whose genome comprises a functional disruption of a selectively-expressed gene. By the phrases “functional disruption” or “functionally disrupted,” it is meant that the gene does not express a biologically-active product. It can be substantially deficient in at least one functional activity coded for by the gene. Expression of a polypeptide can be substantially absent, i.e., essentially undetectable amounts are made. However, polypeptide can also be made, but which is deficient in activity, e.g., where only an amino-terminal portion of the gene product is produced.

The transgenic animal can comprise one or more cells. When substantially all its cells contain the engineered gene, it can be referred to as a transgenic animal “whose genome comprises” the engineered gene. This indicates that the endogenous gene loci of the animal has been modified and substantially all cells contain such modification.

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from the a differentially-regulated gene, etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. A transgenic animal which comprises the functional disruption can also be referred to as a “knock-out” animal, since the biological activity of its a differentially-regulated gene has been “knocked-out.” Knock-outs can be homozygous or heterozygous.

For creating functional disrupted genes, and other gene mutations, homologous recombination technology is of special interest since it allows specific regions of the genome to be targeted. Using homologous recombination methods, genes can be specifically-inactivated, specific mutations can be introduced, and exogenous sequences can be introduced at specific sites. These methods are well known in the art, e.g., as described in the patents above. See, also, Robertson, Biol. Reproduc., 44(2):238-245, 1991. Generally, the genetic engineering is performed in an embryonic stem (ES) cell, or other pluripotent cell line (e.g., adult stem cells, EG cells), and that genetically-modified cell (or nucleus) is used to create a whole organism. Nuclear transfer can be used in combination with homologous recombination technologies.

For example, a differentially-regulated gene locus can be disrupted in mouse ES cells using a positive-negative selection method (e.g., Mansour et al., Nature, 336:348-352, 1988). In this method, a targeting vector can be constructed which comprises a part of the gene to be targeted. A selectable marker, such as neomycin resistance genes, can be inserted into a a differentially-regulated gene exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and germline animals, e.g., animals having somatic and/or germ cells comprising the engineered gene. Homozygous knock-out animals can be obtained from breeding heterozygous knock-out animals. See, e.g., U.S. Pat. No. 6,225,525.

A transgenic animal, or animal cell, lacking one or more functional differentially-regulated genes can be useful in a variety of applications, including, as an animal model for prostate cancer, for drug screening assays, as a source of tissues deficient in said gene activity, and any of the utilities mentioned in any issued U.S. patent on transgenic animals, including, U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. The present invention also relates to non-human, transgenic animal whose genome comprises recombinant a differentially-regulated gene nucleic acid operatively linked to an expression control sequence effective to express said coding sequence, e.g., in prostate. such a transgenic animal can also be referred to as a “knock-in” animal since an exogenous gene has been introduced, stably, into its genome.

A recombinant a differentially-regulated gene nucleic acid refers to a gene which has been introduced into a target host cell and optionally modified, such as cells derived from animals, plants, bacteria, yeast, etc. A recombinant a differentially-regulated gene includes completely synthetic nucleic acid sequences, semi-synthetic nucleic acid sequences, sequences derived from natural sources, and chimeras thereof. “Operable linkage” has the meaning used through the specification, i.e., placed in a functional relationship with another nucleic acid. When a gene is operably linked to an expression control sequence, as explained above, it indicates that the gene (e.g., coding sequence) is joined to the expression control sequence (e.g., promoter) in such a way that facilitates transcription and translation of the coding sequence. As described above, the phrase “genome” indicates that the genome of the cell has been modified. In this case, the recombinant a differentially-regulated gene has been stably integrated into the genome of the animal. The a differentially-regulated gene nucleic acid in operable linkage with the expression control sequence can also be referred to as a construct or transgene.

Any expression control sequence can be used depending on the purpose. For instance, if selective expression is desired, then expression control sequences which limit its expression can be selected. These include, e.g., tissue or cell-specific promoters, introns, enhancers, etc. For various methods of cell and tissue-specific expression, see, e.g., U.S. Pat. Nos. 6,215,040, 6,210,736, and 6,153,427. These also include the endogenous promoter, i.e., the coding sequence can be operably linked to its own promoter. Inducible and regulatable promoters can also be utilized.

The present invention also relates to a transgenic animal which contains a functionally disrupted and a transgene stably integrated into the animals genome. Such an animal can be constructed using combinations any of the above- and below-mentioned methods. Such animals have any of the aforementioned uses, including permitting the knock-out of the normal gene and its replacement with a mutated gene. Such a transgene can be integrated at the endogenous gene locus so that the functional disruption and “knock-in” are carried out in the same step.

In addition to the methods mentioned above, transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Ann. Rev. Genet., 20:465-499, 1986; Askew et al., Mol. Cell. Bio., 13:4115-4124, 1993; Games et al. Nature, 373:523-527, 1995; Valancius and Smithies, Mol. Cell. Bio., 11:1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617,1993; Cibelli et al., Science, 280:1256-1258, 1998. For guidance on recombinase excision systems, see, e.g., U.S. Pat. Nos. 5,626,159, 5,527,695, and 5,434,066. See also, Orban, P. C., et al., “Tissue-and Site-Specific DNA Recombination in Transgenic Mice,” Proc. Natl. Acad. Sci. USA, 89:6861-6865 (1992); O'Gorman, S., et al., “Recombinase-Mediated Gene Activation and Site-Specific Integration in Mammalian Cells,” Science, 251:1351-1355 (1991); Sauer, B., et al., “Cre-stimulated recombination at 1oxP-Containing DNA sequences placed into the mammalian genome,” Polynucleotides Research, 17(1):147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest. 100:169-179; Barlow, C. et al. (1997) Nucl. Acids Res. 25:2543-2545; Araki, K. et al. (1997) Nucl. Acids Res. 25:868-872; Mortensen, R. N. et al. (1992) Mol. Cell. Biol. 12:2391-2395 (G418 escalation method); Lakhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

A polynucleotide according to the present invention can be introduced into any non-human animal, including a non-human mammal, mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-345, 1985), sheep (Hammer et al, Nature, 315:343-345, 1985), cattle, rat, or primate. See also, e.g., Church, 1987, Trends in Biotech. 5:13-19; Clark et al., Trends in Biotech. 5:20-24, 1987); and DePamphilis et al., BioTechniques, 6:662-680, 1988. Transgenic animals can be produced by the methods described in U.S. Pat. No. 5,994,618, and utilized for any of the utilities described therein.

Tissue and Disease

The prostate is a secretory organ surrounding the neck of the bladder and urethra. Its primary function is to produce fluids and other materials necessary for sperm transport and maintenance. Structurally, it has both glandular and nonglandular components. The glandular component is predominantly comprised of ducts and acini responsible for the production and transport prostatic fluids. Epithelial cells are the main identifiable cell found in these regions, primarily of the basal and secretory types, but also endocrine-paracrine and transitional epithelial. The non-glandular component contains the capsular and muscle tissues, which, respectively, hold the organ together and function in fluid discharge. See, e.g., Histology for Pathologists, Sternberg, S. S., editor, Raven Press, NY, 1992, Chapter 40.

The major diseases of the prostate include prostatic hyperplasia (BPH), prostatitis, and prostate cancer (e.g., prostatic adenocarcinoma). BPH is a benign, proliferative disease of the prostatic epithelial cells. While it may cause urinary tract obstruction in some patients, for the most part, it is generally asymptomatic. Prostate cancer, on the other hand, is the most common form of cancer in white males in the United States, occurring predominantly in males over age 50. The first clinical symptoms of prostate cancer are typically urinary disturbances, including painful and more frequent urination. Diagnosis for prostate cancer is usually accomplished using a combination of different procedures. Since the prostate is located next to the rectum, rectal digital examination allows the prostate to be examined manually for the presence of hyperplasia and abnormal tissue masses. Usually, this is the first line of detection. If a palpable mass is observed, a blood specimen can be assayed for prostate-specific antigen (PSA). Very little PSA is present in the blood of a healthy individual, but BPH and prostate cancer can cause large amounts of PSA to be released into the blood, indicating the presence of diseased tissue. Definitive diagnosis is generally accomplished by biopsy of the prostate tissue. The most common scale of assessing prostate pathology is the Gleason grading system. See, e.g., Bostwick, Am. J. Clin. Path., 102: s38-s56, 1994. Once the cancer is identified, staging can assess the size, location, and extent of the cancer. Several different staging scales are commonly used, including stages A-D, and Tumor-Nodes-Metastases (TNM). For treatment, diagnosis, staging, etc., of prostate conditions, methods can be carried out analogously to, and in combination with, U.S. Pat. Nos. 6,107,090; 6,057,116; 6,034,218; 6,004,267; 5,919,638; 5,882,864; 5,763,202; 5,747,264; 5,688,649; 5,552,277.

Detection and staging of prostate disease can be accomplished using polynucleotide probes, antibodies, specific-binding partners, etc., in accordance with the present invention. Antibodies and other probes can be used in vitro on biopsied tissue (e.g., as markers to identify and characterize premalignant tissues and cells, intraepithelial neoplasia, adenocarcinoma, atypical adenomatous hyperplasia, and other neoplasias and carcinomas), in blood (whole, plasma, serum, etc.) and other bodily fluid (semen, urine, stool, etc.) and tissue samples, e.g., to identify metastatic and rogue cells, as well as for in vivo imaging according to conventional methodologies. The probes and markers can be useful to identify the ancestry of a cancer and its tissue of origin. These markers and probes can be used alone, or together with other known tests and genes, such as those disclosed in the publications cited above and below. Differential diagnosis can be enhanced when Gleason grading and TNM, for example, are used in conjunction with methods of detection as described herein. Together, these methods provide more accurate disease diagnosis, disease progression, and other information useful for determining therapy and prognosis of the cancer.

A number of genes and gene products have been identified which are associated with prostate cancer metastasis and/or progression, e.g., PSA, KAI1(shows decreased expression in metastatic cells; Dong et al., Science, 268:884-6, 1995), D44 isoforms (differentially-regulated during carcinoma progression; Noordzij et al., Clin. Cancer Res., 3:805-15, 1997), p53 (Effert et al., J. Urol., 150:257-61, 1993), Rb, CDKN2, E-cadherin, PTEN (Hamilton et al., Br. J. Cancer, 82:1671-6, 2000), bcl-2, prostatic acid phosphatase (PAP), prostate specific membrane antigen (e.g., U.S. Pat. No. 6,107,090), and other oncogenes and tumor suppressor genes. See, also, Myers and Grizzle, Eur. Urol., 30:153-166, 1996, for other biomarkers associated with prostatic carcinoma, such as PCNA, p185-erbB-2, p180erbB-3, TAG-72, nm23-H1 and FASE. Such markers can be used in combination with the methods of the present invention to facilitate identifying, grading, staging, prognostication, etc, of conditions and diseases of the prostate.

No Open Reading Frame

In addition to their use as tissue-selective markers and probes, the noncoding RNAs of the present invention can have one or more of the functions associated with known noncoding RNAs, including the functions displayed by, e.g., His-i (Askew et al., Oncogene, 6:2041-2047, 1991), Bic (Tam et al., Mol. Cell. Biol., 17:1490-1502, 1997), H19 (Leighton et al., Nature, 375:34-39, 1995; Cui et al., Cancer Res., 67:4469-4473, 1997; Frevel et al., J. Biol. Chem., 274:29331-29340, 1999), XIST (Herzing et al., Nature, 386:272-275, 1997); 3′ UTR (Goodwin et al., 1993; Rastinejad et al., Cell, 75:1107-1117, 1993; Rastinejad and Blau, Cell, 72:903-917, 1993); introns RNA (Cech, Ann. Rev. Biochem., 59:543-568, 1990); IPW (Wevrick et al., Hum. Mol. Genet., 3:1877-1882, 1994), NTT (Liu et al., Genomics, 39:171-184, 1997); 7H4 Velleca et al., Mol. Cell. Biol., 14:7096-7104, 1994); and Tsix (Lee, Cell, 103:17-27, 2000). See, also, Askew and Xu, Histol. Histopathol., 14:235-241, 1999. Noncoding RNAs can possess a functional role in a variety of biological processes, including, but not limited to, oncogenesis, genomic imprinting (e.g., regulation and maintenance), as a tumor suppressor, gene suppression, regulation of translation, activation of tissue-specific promoters, modulation of cell growth and differentiation, RNA self-splicing, as a DNA methylation site, monoalleic exclusion of imprinted genes, etc.

PR33a and PR33b contain Alu-type sequences in anti-sense orientation, making them useful, e.g., as probes to detect and quantitate Alu sequences, and as modulators of expressed Alu sequences. For instance, the PR33 family can be used to determine the presence and amounts of endogenous small cytoplasmic Alu sequences (scAlu) and full-length Alu (flAlu) sequences in a tissue sample. In addition, the family of PR33 transcripts can also act as modulators of endogenous Alu sequences, such as the Alu sequences present in the 7SL RNA of the signal-recognition particle (SRP), as well as scALu and flAlu sequences. PR33 transcripts can act as antagonists, inhibiting the formation of the SRP by forming double-stranded structures with the 7SL RNA at regions of complementarity, or, lowering the physiologic levels of endogenous scAlu and flAlu by the same base-pairing mechanisms.

Therapeutics

Selective polynucleotides, polypeptides, and specific-binding partners thereto, can be utilized in therapeutic applications, especially to treat diseases and conditions of the prostate. Useful methods include, but not limited to, immunotherapy (e.g., using specific-binding partners to polypeptides), vaccination (e.g., using a selective polypeptide or a naked DNA encoding such polypeptide, protein or polypeptide replacement therapy, gene therapy (e.g., germ-line correction, antisense), etc.

Various immunotherapeutic approaches can be used. For instance, unlabeled antibody that specifically recognizes a prostate-specific antigen can be used to stimulate the body to destroy or attack the cancer, cause down-regulation, complement-mediated lysis, inhibit cell growth, etc., of target cells which display the antigen, e.g., analogously to how c-erbB-2 antibodies are used to treat breast cancer. In addition, antibody can be labeled or conjugated to enhance its deleterious effect, e.g., with radionuclides and other energy emitting entitities, toxins, such as ricin, exotoxin A (ETA), and diphtheria, cytotoxic or cytostatic agents, immunomodulators, chemotherapeutic agents, etc. See, e.g., U.S. Pat. No. 6,107,090.

Delivery of therapeutic agents can be achieved according to any effective method, including, liposomes, viruses, plasmid vectors, bacterial delivery systems, orally, aerosol systemically, etc.

Other

A polynucleotide, probe, polypeptide, antibody, specific-binding partner, etc., according to the present invention can be isolated. The term “isolated” means that the material is in a form in which it is not found in its original environment or in nature, e.g., more concentrated, more purified, separated from component, etc. An isolated polynucleotide includes, e.g., a polynucleotide having the sequenced separated from the chromosomal DNA found in a living animal, e.g., as the complete gene, a transcript, or a cDNA. This polynucleotide can be part of a vector or inserted into a chromosome (by specific gene-targeting or by random integration at a position other than its normal position) and still be isolated in that it is not in a form that is found in its natural environment. A polynucleotide, polypeptide, etc., of the present invention can also be substantially purified. By substantially purified, it is meant that polynucleotide or polypeptide is separated and is essentially free from other polynucleotides or polypeptides, i.e., the polynucleotide or polypeptide is the primary and active constituent. A polynucleotide can also be a recombinant molecule. By “recombinant,” it is meant that the polynucleotide is an arrangement or form which does not occur in nature. For instance, a recombinant molecule comprising a promoter sequence would not encompass the naturally-occurring gene, but would include the promoter operably linked to a coding sequence not associated with it in nature, e.g., a reporter gene, or a truncation of the normal coding sequence.

The term “marker” is used herein to indicate a means for detecting or labeling a target. A marker can be a polynucleotide (usually referred to as a “probe”), polypeptide (e.g., an antibody conjugated to a detectable label), PNA, or any effective material.

The topic headings set forth above are meant as guidance where certain information can be found in the application, but are not intended to be the only source in the application where information on such topic can be found.

For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

The preceding description, utilize the present invention to its fullest extent. The preceding preferred specific embodiments are, therefore, to be construed as merely illustrative, and not limiting the remainder of the disclosure in any way whatsoever. The entire disclosure of all applications, patents, publications, GenBank accession numbers, sequence disclosures, etc., cited above are hereby incorporated by reference in their entirety.

1. An isolated polynucleotide which is selectively expressed in prostate, which is:
PR33a as set forth in SEQ ID NO. 1, PR33b as set forth in SEQ ID NO. 3, PRB008 as set forth in SEQ ID NO. 4, a polynucleotide having 95% sequence identity thereto, or a complement thereto.

2. An isolated polynucleotide of claim 1, which is PR33a as set forth in SEQ ID NO. 1.

3. An isolated polynucleotide of claim 1, which is PR33b as set forth in SEQ ID NO.3.

4. An isolated polynucleotide of claim 1, which is PRB008 as set forth in SEQ ID NO. 4.

5. An isolated polynucleotide probe for prostate, comprising:
SEQ ID NOS 7, 8, 9, 10, 11, 12, 13, 14, 15, 16, or a complement thereto.

6. An isolated probe of claim 5, which consists essentially of SEQ ID NOS. 7, 8, 9, 10, 11, 12, 13, 14, 15, 16, or a complement thereto.

7. A method of detecting prostate tissue in a sample comprising nucleic acid, comprising:
contacting said sample with a polynucleotide probe under conditions effective for said probe to hybridize specifically to a nucleic acid of claim 1 in said sample, and
detecting the presence or absence of probe hybridized to said nucleic acid in said sample,
wherein said probe is a polynucleotide which is PR33a as set forth in SEQ ID NO. 1, PR33b as set forth in SEQ ID NO. 3, PRB008 as set forth in SEQ ID NO. 4, complements thereto, a polynucleotide having at least 95% sequence identity thereto, or effective specific fragments thereof.

8. A method of claim 7, wherein said probe is a contiguous sequence of at least 8 nucleotides selected from the sequence set forth in SEQ ID NOS. 1, 3, 4, or complements thereto.

9. A method of claim 7, wherein said probe is selected from SEQ ID NOS. 7-16, or a complement thereto.

10. A method of claim 7, wherein said detecting is performed by Northern blot analysis, polymerase chain reaction (PCR), reverse transcriptase PCR, RACE PCR, or in situ hybridization.

11. A method of claim 7, wherein said sample is blood, normal prostate, or prostate cancer.

12. A method of retrieving prostate-specific gene sequences from a computer-readable medium, comprising:
selecting a gene expression profile that specifies that said gene is selectively expressed in prostate, and
retrieving prostate-specific gene sequences, where the gene sequences comprise the sequences of claim 1.

13. A method of claim 12, wherein said gene has the nucleotide sequence set forth in SEQ ID NOS. 1, 3, 4, or complements thereto.

14. A method of identifying specific-binding partners for prostate-specific polynucleotides comprising:
contacting a PR33a, PR33b, or PRB008 polynucleotide of claim 1 with a sample comprising a specific-binding partner under conditions effective for said partner to bind to polynucleotide, and
detecting the presence or absence of binding between said polynucleotide and said specific-binding partner.

15. A method of claim 14, wherein said detecting is performed using a gel band-shift assay.

16. A computer-readable storage medium, consisting essentially of,
polynucleotide sequences of claim 1.

17. A storage medium of claim 16, wherein said gene has a nucleotide sequence set forth in SEQ ID NO. 1, 3, or 4.

18. An array of polynucleotide probes, comprising:
nucleic acid probes selective for prostate-selective genes comprising (a) PR33a or PR33b, and (b) PRB008,
wherein said probes are selected from PR33a as set forth in SEQ ID NO. 1, PR33b as set forth in SEQ ID NO. 3, PRB008 as set forth in SEQ ID NO. 4, or complements thereto, and said probe is a contiguous sequence of at least 8 nucleotides.

19. A recombinant polynucleotide molecule comprising, a promoter sequence of SEQ ID NO 6.

20. A recombinant polynucleotide molecule of claim 19, further comprising a coding sequence operably linked to said promoter.
